SEQUENCE LISTING (11 sequences)

SEQ ID NO: 1 - protein, 1273 aa, Artificial Sequence
  amino acid sequence of S protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 3822 bp, Artificial Sequence
  S protein coding sequence, S-FL-wt
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 3 - DNA, 3822 bp, Artificial Sequence
  S protein coding sequence, S-FL-opt
atgttcgtgttcctggtgctcctccctctcgtgtcttctcagtgcgtgaacctgaccacacggacccagctgccacccgcttacaccaactccttcacaagaggcgtgtactaccccgacaaggtgttccggtcttctgtgctccactctacccaggacctgttcctgccattcttctctaacgtgacatggttccacgctatccacgtgtctggcaccaacggcacaaagagattcgacaaccccgtgctcccattcaacgacggcgtgtacttcgctagcacagagaagtccaacatcatccggggctggatcttcggcaccacactggactctaagacccagtccctcctcatcgtgaacaacgccacaaacgtggtgatcaaggtgtgcgagttccagttctgcaacgacccattcctgggcgtgtactaccacaagaacaacaagtcttggatggagagcgagttccgggtgtactctagcgctaacaactgcacattcgagtacgtgtctcagccattcctcatggacctcgagggcaagcagggcaacttcaagaacctgagggagttcgtgttcaagaacatcgacggctacttcaagatctactctaagcacaccccaatcaacctggtgagggacctgcctcagggcttctccgctctcgagccactggtggacctgccaatcggcatcaacatcacccggttccagacactcctcgccctccaccggtcttacctgaccccaggcgactctagcagcggctggaccgccggcgccgccgcttactacgtgggctacctgcagcctaggacattcctcctgaagtacaacgagaacggcacaatcaccgacgctgtggactgcgccctcgacccactgagcgagacaaagtgcacactgaagtctttcacagtggagaagggcatctaccagacatctaacttcagagtgcagcctacagagtctatcgtgagattcccaaacatcacaaacctgtgccctttcggcgaggtgttcaacgctacacggttcgcttctgtgtacgcttggaaccggaagcggatctctaactgcgtggccgactactctgtgctgtacaactccgcctctttctccacattcaagtgctacggcgtgagcccaacaaagctgaacgacctgtgcttcacaaacgtgtacgccgactctttcgtgatccggggcgacgaggtgaggcagatcgctccaggccagacaggcaagatcgccgactacaactacaagctccccgacgacttcacaggctgcgtgatcgcttggaactctaacaacctggactctaaggtgggcggcaactacaactacctgtacagactgttccggaagtctaacctgaagcctttcgagcgggacatctctaccgagatctaccaggccggcagcaccccttgcaacggcgtggagggcttcaactgctacttcccactgcagtcttacggcttccagcctacaaacggcgtgggctaccagccataccgggtggtggtgctgtccttcgagctcctccacgcccccgctacagtgtgcggcccaaagaagtccacaaacctcgtgaagaacaagtgcgtgaacttcaacttcaacggcctgacaggcacaggcgtgctcacagagtctaacaagaagttcctccctttccagcagttcggccgggacatcgccgacaccaccgacgccgtgagggaccctcagacactcgagatcctcgacatcaccccttgctctttcggcggcgtgtctgtgatcacccccggcacaaacacatctaaccaggtggctgtgctgtaccaggacgtgaactgcaccgaggtgccagtggctatccacgccgaccagctcacccctacatggagggtgtacagcacaggctctaacgtgttccagacgagggccggctgcctcatcggcgccgagcacgtgaacaactcttacgagtgcgacatcccaatcggcgctggcatctgcgcttcttaccagacccagacaaacagccctaggagagctaggtccgtggctagccagtccatcatcgcttacaccatgtctctgggcgctgagaactctgtggcttactctaacaactctatcgctatccctacaaacttcacaatctctgtgaccaccgagatcctgccagtgtctatgacaaagacatccgtggactgcaccatgtacatctgcggcgacagcaccgagtgcagcaacctgctcctgcagtacggctctttctgcacacagctgaaccgggccctcaccggcatcgccgtggagcaggacaagaacacccaggaggtgttcgctcaggtgaagcagatctacaagaccccccctatcaaggacttcggcggcttcaacttctctcagatcctccccgaccctagcaagcctagcaagcggtctttcatcgaggacctcctgttcaacaaggtgacactcgctgacgctggcttcatcaagcagtacggcgactgcctgggcgacatcgccgctagagacctgatctgcgctcagaagttcaacggcctcacagtgctgcccccactcctgaccgacgagatgatcgctcagtacaccagcgccctcctcgctggcacaatcacatctggctggacattcggcgccggcgccgccctgcagatcccattcgccatgcagatggcttaccggttcaacggcatcggcgtgacccagaacgtgctgtacgagaaccagaagctgatcgctaaccagttcaactccgctatcggcaagatccaggactccctgtctagcaccgctagcgccctgggcaagctccaggacgtggtgaaccagaacgcccaggccctgaacacactggtgaagcagctgtcttctaacttcggcgctatcagctctgtgctcaacgacatcctgtctagactcgacaaggtggaggccgaggtgcagatcgacagactgatcacaggcagactgcagtccctccagacatacgtgacccagcagctcatccgggccgctgagatcagagctagcgctaacctcgccgctaccaagatgagcgagtgcgtgctgggccagtctaagcgggtggacttctgcggcaagggctaccacctcatgtctttccctcagtccgcccctcacggcgtggtgttcctccacgtgacatacgtgccagctcaggagaagaacttcaccaccgccccagctatctgccacgacggcaaggcccacttcccacgggagggcgtgttcgtgtctaacggcacccactggttcgtgacacagcggaacttctacgagcctcagatcatcaccacagacaacacattcgtgtccggcaactgcgacgtggtgatcggcatcgtgaacaacacagtgtacgacccactgcagcccgagctcgactctttcaaggaggagctcgacaagtacttcaagaaccacaccagccccgacgtggacctgggcgacatctctggcatcaacgcctctgtggtgaacatccagaaggagatcgacagactgaacgaggtggctaagaacctgaacgagtccctgatcgacctgcaggagctgggcaagtacgagcagtacatcaagtggccttggtacatctggctgggcttcatcgctggcctgatcgctatcgtgatggtgacaatcatgctgtgctgcatgacctcttgctgctcttgcctgaagggctgctgctcttgcggctcttgctgcaagttcgacgaggacgactccgagcccgtgctgaagggcgtgaagctccactacacatga

SEQ ID NO: 4 - DNA, 3642 bp, Artificial Sequence
  truncated S protein coding sequence, S-dTM-opt
atgttcgtgttcctggtgctcctccctctcgtgtcttctcagtgcgtgaacctgaccacacggacccagctgccacccgcttacaccaactccttcacaagaggcgtgtactaccccgacaaggtgttccggtcttctgtgctccactctacccaggacctgttcctgccattcttctctaacgtgacatggttccacgctatccacgtgtctggcaccaacggcacaaagagattcgacaaccccgtgctcccattcaacgacggcgtgtacttcgctagcacagagaagtccaacatcatccggggctggatcttcggcaccacactggactctaagacccagtccctcctcatcgtgaacaacgccacaaacgtggtgatcaaggtgtgcgagttccagttctgcaacgacccattcctgggcgtgtactaccacaagaacaacaagtcttggatggagagcgagttccgggtgtactctagcgctaacaactgcacattcgagtacgtgtctcagccattcctcatggacctcgagggcaagcagggcaacttcaagaacctgagggagttcgtgttcaagaacatcgacggctacttcaagatctactctaagcacaccccaatcaacctggtgagggacctgcctcagggcttctccgctctcgagccactggtggacctgccaatcggcatcaacatcacccggttccagacactcctcgccctccaccggtcttacctgaccccaggcgactctagcagcggctggaccgccggcgccgccgcttactacgtgggctacctgcagcctaggacattcctcctgaagtacaacgagaacggcacaatcaccgacgctgtggactgcgccctcgacccactgagcgagacaaagtgcacactgaagtctttcacagtggagaagggcatctaccagacatctaacttcagagtgcagcctacagagtctatcgtgagattcccaaacatcacaaacctgtgccctttcggcgaggtgttcaacgctacacggttcgcttctgtgtacgcttggaaccggaagcggatctctaactgcgtggccgactactctgtgctgtacaactccgcctctttctccacattcaagtgctacggcgtgagcccaacaaagctgaacgacctgtgcttcacaaacgtgtacgccgactctttcgtgatccggggcgacgaggtgaggcagatcgctccaggccagacaggcaagatcgccgactacaactacaagctccccgacgacttcacaggctgcgtgatcgcttggaactctaacaacctggactctaaggtgggcggcaactacaactacctgtacagactgttccggaagtctaacctgaagcctttcgagcgggacatctctaccgagatctaccaggccggcagcaccccttgcaacggcgtggagggcttcaactgctacttcccactgcagtcttacggcttccagcctacaaacggcgtgggctaccagccataccgggtggtggtgctgtccttcgagctcctccacgcccccgctacagtgtgcggcccaaagaagtccacaaacctcgtgaagaacaagtgcgtgaacttcaacttcaacggcctgacaggcacaggcgtgctcacagagtctaacaagaagttcctccctttccagcagttcggccgggacatcgccgacaccaccgacgccgtgagggaccctcagacactcgagatcctcgacatcaccccttgctctttcggcggcgtgtctgtgatcacccccggcacaaacacatctaaccaggtggctgtgctgtaccaggacgtgaactgcaccgaggtgccagtggctatccacgccgaccagctcacccctacatggagggtgtacagcacaggctctaacgtgttccagacgagggccggctgcctcatcggcgccgagcacgtgaacaactcttacgagtgcgacatcccaatcggcgctggcatctgcgcttcttaccagacccagacaaacagccctaggagagctaggtccgtggctagccagtccatcatcgcttacaccatgtctctgggcgctgagaactctgtggcttactctaacaactctatcgctatccctacaaacttcacaatctctgtgaccaccgagatcctgccagtgtctatgacaaagacatccgtggactgcaccatgtacatctgcggcgacagcaccgagtgcagcaacctgctcctgcagtacggctctttctgcacacagctgaaccgggccctcaccggcatcgccgtggagcaggacaagaacacccaggaggtgttcgctcaggtgaagcagatctacaagaccccccctatcaaggacttcggcggcttcaacttctctcagatcctccccgaccctagcaagcctagcaagcggtctttcatcgaggacctcctgttcaacaaggtgacactcgctgacgctggcttcatcaagcagtacggcgactgcctgggcgacatcgccgctagagacctgatctgcgctcagaagttcaacggcctcacagtgctgcccccactcctgaccgacgagatgatcgctcagtacaccagcgccctcctcgctggcacaatcacatctggctggacattcggcgccggcgccgccctgcagatcccattcgccatgcagatggcttaccggttcaacggcatcggcgtgacccagaacgtgctgtacgagaaccagaagctgatcgctaaccagttcaactccgctatcggcaagatccaggactccctgtctagcaccgctagcgccctgggcaagctccaggacgtggtgaaccagaacgcccaggccctgaacacactggtgaagcagctgtcttctaacttcggcgctatcagctctgtgctcaacgacatcctgtctagactcgacaaggtggaggccgaggtgcagatcgacagactgatcacaggcagactgcagtccctccagacatacgtgacccagcagctcatccgggccgctgagatcagagctagcgctaacctcgccgctaccaagatgagcgagtgcgtgctgggccagtctaagcgggtggacttctgcggcaagggctaccacctcatgtctttccctcagtccgcccctcacggcgtggtgttcctccacgtgacatacgtgccagctcaggagaagaacttcaccaccgccccagctatctgccacgacggcaaggcccacttcccacgggagggcgtgttcgtgtctaacggcacccactggttcgtgacacagcggaacttctacgagcctcagatcatcaccacagacaacacattcgtgtccggcaactgcgacgtggtgatcggcatcgtgaacaacacagtgtacgacccactgcagcccgagctcgactctttcaaggaggagctcgacaagtacttcaagaaccacaccagccccgacgtggacctgggcgacatctctggcatcaacgcctctgtggtgaacatccagaaggagatcgacagactgaacgaggtggctaagaacctgaacgagtccctgatcgacctgcaggagctgggcaagtacgagcagtacatcaagtggccttga

SEQ ID NO: 5 - DNA, 61 bp, Artificial Sequence
  oligmer for amplification of S-FL-opt insert
tccatgggtcttttctgcagtcaccgtccaagcttgcaatcgccaccatgttcgtgttcct

SEQ ID NO: 6 - DNA, 41 bp, Artificial Sequence
  oligmer for amplification of S-FL-opt insert
gggattgcgaggatccttatcatgtgtagtggagcttcacg

SEQ ID NO: 7 - protein, 685 aa, Artificial Sequence
  amino acid sequence of S1 subunit
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 8 - DNA, 17 bp, Artificial Sequence
  probe for SARS-CoV2 N (nucleocapsid) gene
ttgctgcttgacagatt

SEQ ID NO: 9 - DNA, 22 bp, Artificial Sequence
  forward primer for SARS-CoV2 N (nucleocapsid) gene
ggggaacttctcctgctagaat

SEQ ID NO: 10 - DNA, 22 bp, Artificial Sequence
  reverse primer for SARS-CoV2 N (nucleocapsid) gene
cagacattttgctctcaagctg

SEQ ID NO: 11 - DNA, 8515 bp, Artificial Sequence
  pCW1093 full length sequence
ctgacgtcgcggtcgacaatattggctattggccattgcatacgttgtatctatatcataatatgtacatttatattggctcatgtccaatatgaccgccatgttgacattagttattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccaagcttgcaatcgccaccatgttcgtgttcctggtgctcctccctctcgtgtcttctcagtgcgtgaacctgaccacacggacccagctgccacccgcttacaccaactccttcacaagaggcgtgtactaccccgacaaggtgttccggtcttctgtgctccactctacccaggacctgttcctgccattcttctctaacgtgacatggttccacgctatccacgtgtctggcaccaacggcacaaagagattcgacaaccccgtgctcccattcaacgacggcgtgtacttcgctagcacagagaagtccaacatcatccggggctggatcttcggcaccacactggactctaagacccagtccctcctcatcgtgaacaacgccacaaacgtggtgatcaaggtgtgcgagttccagttctgcaacgacccattcctgggcgtgtactaccacaagaacaacaagtcttggatggagagcgagttccgggtgtactctagcgctaacaactgcacattcgagtacgtgtctcagccattcctcatggacctcgagggcaagcagggcaacttcaagaacctgagggagttcgtgttcaagaacatcgacggctacttcaagatctactctaagcacaccccaatcaacctggtgagggacctgcctcagggcttctccgctctcgagccactggtggacctgccaatcggcatcaacatcacccggttccagacactcctcgccctccaccggtcttacctgaccccaggcgactctagcagcggctggaccgccggcgccgccgcttactacgtgggctacctgcagcctaggacattcctcctgaagtacaacgagaacggcacaatcaccgacgctgtggactgcgccctcgacccactgagcgagacaaagtgcacactgaagtctttcacagtggagaagggcatctaccagacatctaacttcagagtgcagcctacagagtctatcgtgagattcccaaacatcacaaacctgtgccctttcggcgaggtgttcaacgctacacggttcgcttctgtgtacgcttggaaccggaagcggatctctaactgcgtggccgactactctgtgctgtacaactccgcctctttctccacattcaagtgctacggcgtgagcccaacaaagctgaacgacctgtgcttcacaaacgtgtacgccgactctttcgtgatccggggcgacgaggtgaggcagatcgctccaggccagacaggcaagatcgccgactacaactacaagctccccgacgacttcacaggctgcgtgatcgcttggaactctaacaacctggactctaaggtgggcggcaactacaactacctgtacagactgttccggaagtctaacctgaagcctttcgagcgggacatctctaccgagatctaccaggccggcagcaccccttgcaacggcgtggagggcttcaactgctacttcccactgcagtcttacggcttccagcctacaaacggcgtgggctaccagccataccgggtggtggtgctgtccttcgagctcctccacgcccccgctacagtgtgcggcccaaagaagtccacaaacctcgtgaagaacaagtgcgtgaacttcaacttcaacggcctgacaggcacaggcgtgctcacagagtctaacaagaagttcctccctttccagcagttcggccgggacatcgccgacaccaccgacgccgtgagggaccctcagacactcgagatcctcgacatcaccccttgctctttcggcggcgtgtctgtgatcacccccggcacaaacacatctaaccaggtggctgtgctgtaccaggacgtgaactgcaccgaggtgccagtggctatccacgccgaccagctcacccctacatggagggtgtacagcacaggctctaacgtgttccagacgagggccggctgcctcatcggcgccgagcacgtgaacaactcttacgagtgcgacatcccaatcggcgctggcatctgcgcttcttaccagacccagacaaacagccctaggagagctaggtccgtggctagccagtccatcatcgcttacaccatgtctctgggcgctgagaactctgtggcttactctaacaactctatcgctatccctacaaacttcacaatctctgtgaccaccgagatcctgccagtgtctatgacaaagacatccgtggactgcaccatgtacatctgcggcgacagcaccgagtgcagcaacctgctcctgcagtacggctctttctgcacacagctgaaccgggccctcaccggcatcgccgtggagcaggacaagaacacccaggaggtgttcgctcaggtgaagcagatctacaagaccccccctatcaaggacttcggcggcttcaacttctctcagatcctccccgaccctagcaagcctagcaagcggtctttcatcgaggacctcctgttcaacaaggtgacactcgctgacgctggcttcatcaagcagtacggcgactgcctgggcgacatcgccgctagagacctgatctgcgctcagaagttcaacggcctcacagtgctgcccccactcctgaccgacgagatgatcgctcagtacaccagcgccctcctcgctggcacaatcacatctggctggacattcggcgccggcgccgccctgcagatcccattcgccatgcagatggcttaccggttcaacggcatcggcgtgacccagaacgtgctgtacgagaaccagaagctgatcgctaaccagttcaactccgctatcggcaagatccaggactccctgtctagcaccgctagcgccctgggcaagctccaggacgtggtgaaccagaacgcccaggccctgaacacactggtgaagcagctgtcttctaacttcggcgctatcagctctgtgctcaacgacatcctgtctagactcgacaaggtggaggccgaggtgcagatcgacagactgatcacaggcagactgcagtccctccagacatacgtgacccagcagctcatccgggccgctgagatcagagctagcgctaacctcgccgctaccaagatgagcgagtgcgtgctgggccagtctaagcgggtggacttctgcggcaagggctaccacctcatgtctttccctcagtccgcccctcacggcgtggtgttcctccacgtgacatacgtgccagctcaggagaagaacttcaccaccgccccagctatctgccacgacggcaaggcccacttcccacgggagggcgtgttcgtgtctaacggcacccactggttcgtgacacagcggaacttctacgagcctcagatcatcaccacagacaacacattcgtgtccggcaactgcgacgtggtgatcggcatcgtgaacaacacagtgtacgacccactgcagcccgagctcgactctttcaaggaggagctcgacaagtacttcaagaaccacaccagccccgacgtggacctgggcgacatctctggcatcaacgcctctgtggtgaacatccagaaggagatcgacagactgaacgaggtggctaagaacctgaacgagtccctgatcgacctgcaggagctgggcaagtacgagcagtacatcaagtggccttggtacatctggctgggcttcatcgctggcctgatcgctatcgtgatggtgacaatcatgctgtgctgcatgacctcttgctgctcttgcctgaagggctgctgctcttgcggctcttgctgcaagttcgacgaggacgactccgagcccgtgctgaagggcgtgaagctccactacacatgataaggatcctcgcaatccctaggaggattaggcaagggcttgagctcacgctcttgtgagggacagaaatacaatcaggggcagtatatgaatactccatggagaaacccagatctacgtatgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgacagctcgagctaggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcgcggccgcgatccgtcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtctagcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcatgctagaactcttcctttttcaatattattgaagcatttatcagggttattgtctcatgagcggatacatatttgaatgtatttagaaaaataaacaaataggggttccgcgcacatttccccgaaaagtgccac

(19) United States
(12) Patent Application Publication
(10) Pub. No.: US 2024/0016920 A1
(43) Pub. Date: Jan. 18, 2024

(54) IMMUNITY AND PROTECTION OF SARS-COV-2 DNA AND PROTEIN VACCINE

(71) Applicants: Shan LU, Worcester, MA (US); INSTITUTE OF MEDICAL BIOLOGY, CHINESE ACADEMY OF MEDICAL SCIENCES & PEKING UNION MEDICAL COLLEGE, Kunming, Yunnan (CN)

(72) Inventors: Wei CUN, Yunnan (CN); Shan LU, Worcester, MA (US); Qihan LI, Yunnan (CN); Hongjian XIAO, Yunnan (CN); Yanwei BI, Yunnan (CN); Yuzhong LI, Yunnan (CN); Xiaojuan LIU, Yunnan (CN); Zhihua LI, Yunnan (CN)

(21) Appl. No.: 18/254,373

(22) PCT Filed: Nov. 24, 2021
(86) PCT No.: PCT/CN2021/132703

§ 371 Date: May 24, 2023

(30) Foreign Application Priority Data
Nov. 24, 2020 (WO) PCT/CN2020/131098

(51) Int. Cl. A61K 39/215; C07K 14/165; A61P 31/14

(52) U.S. Cl. CPC A61K 39/215; C07K 14/165; A61P 31/14; A61K 2039/53

(57) ABSTRACT

Provided are a DNA vaccine against SARS-CoV-2 virus infection in a subject which comprises a codon optimized polynucleotide sequence encoding a polypeptide of the SARS-CoV-2 virus. Also provided are a vaccine combination against SARS-CoV-2 vims infection, which comprises said DNA vaccine and an antigen peptide vaccine. The vaccine combination is able to confer a full protection against the SARS-CoV-2 vims infection in NHP studies.

### CROSS-REFERENCE TO RELATED APPLICATION

This application claims priority to PCT patent application PCT/CN2020/131098, filed Nov. 24, 2020, the content of which is incorporated herein by reference.

### FIELD OF THE INVENTION

The present disclosure relates to a vaccine against SARS-CoV-2 virus infection, and, especially, relates to a vaccine combination against SARS-CoV-2 virus infection comprising a DNA vaccine and an antigen peptide vaccine.

### BACKGROUND

The COVID-19 pandemic has caused over 30 million cases including 1.2 million deaths globally (1). While public health measures such as social distancing has played important roles in controlling local outbreaks, the continued spreading of COVID-19 pandemic to additional populations including those living in remote and underdeveloped areas would only extend the future threat (2-6). In addition, new waves of transmissions are occurring in many countries even after the original outbreaks came down (7, 8). More definitive large scale public health measures like vaccines are the only hope to achieve the full global control (9-12).

Currently there are at least a dozen COVID-19 vaccines have entered Phase III clinical studies to establish their efficacy before the wide public use (13). Several leading candidates are using novel vaccine platforms such as viral vector (14-18) or mRNA (19-23) approaches. No human preventive vaccines using these approaches have been formally licensed with efficacy clinical studies in the past. One other major type of COVID-19 vaccines is the inactivated vaccine approach (24-28) which is linked to possible adverse events observed with such type of vaccines in the past (29, 30). There are also biosafety issues related to the need of producing large stocks of live SARS-CoV-2 viruses before inactivation. Overall viral vector, nucleic acid or inactivated vaccines are not considered highly immunogenic based on the past experience. At the same time, it is reported that SARS-CoV-2 infection may not lead to high level immune responses and some recovered patients may be re-infected again by the same virus (31-33). Therefore, it is highly desirable to develop COVID-19 vaccines that are highly immunogenic and the elicited immunity is long lasting.

### SUMMARY

In one aspect, the present disclosure provides a DNA vaccine for use in a subject against SARS-CoV-2 virus infection, which comprises a polynucleotide sequence encoding a polypeptide of the SARS-CoV-2 virus, wherein the polynucleotide sequence is codon optimized for expression in the subject.

In some embodiments, the polypeptide is the SARS-CoV-2 spike protein or comprises at least a conserved moiety of the SARS-CoV-2 spike protein.

In some embodiments, the polypeptide comprises the receptor-binding domain (RBD) of the spike protein.

In some embodiments, the subject is a human being.

In some embodiments, the DNA vaccine is a plasmid constructed from plasmid pSW3891.

In some embodiments, the polynucleotide sequence comprises a sequence of SEQ ID NO: 3 or 4.

In another aspect, the present disclosure provides a method for preventing or treating SARS-CoV-2 virus infection in a subject, which comprises administering to the subject an effective amount of an DNA vaccine, wherein the DNA vaccine comprises a polynucleotide sequence encoding a polypeptide of the SARS-CoV-2 virus, and the polynucleotide sequence is codon optimized for expression in the subject.

In some embodiments, the polypeptide is the SARS-CoV-2 spike protein or comprises at least a conserved moiety of the SARS-CoV-2 spike protein.

In some embodiments, the polypeptide comprises RBD of the spike protein.

In some embodiments, the subject is a human being.

In some embodiments, the DNA vaccine is a plasmid constructed from plasmid pSW3891.

In some embodiments, the polynucleotide sequence comprises a sequence of SEQ ID NO: 3 or 4.

In another aspect, the present disclosure provides a vaccine combination for use in a subject against SARS-CoV-2 virus infection, which comprises:
1) a DNA vaccine comprising a polynucleotide sequence encoding a polypeptide of the SARS-CoV-2 virus; and
2) an antigen peptide vaccine, wherein the antigen peptide is an antigen peptide of the SARS-CoV-2 virus.

In some embodiments, the polynucleotide sequence is codon optimized for expression in the subject.

In some embodiments, the polypeptide is the SARS-CoV-2 spike protein or comprises at least a conserved moiety of the SARS-CoV-2 spike protein.

In some embodiments, the polypeptide comprises RBD of the spike protein.

In some embodiments, the subject is a human being.

In some embodiments, the DNA vaccine is a plasmid constructed from plasmid pSW3891.

In some embodiments, the polynucleotide sequence comprises a sequence of SEQ ID NO: 3 or 4.

In some embodiments, the antigen peptide comprises at least a conserved moiety of the SARS-CoV-2 spike protein.

In some embodiments, the antigen peptide comprises RBD of the spike protein.

In some embodiments, the antigen peptide is the S1 subunit of the spike protein.

In some embodiments, the antigen peptide comprises an amino acid sequence of SEQ ID NO: 7 or a functional variant with sequence identity of 80% or more to SEQ ID NO: 7.

In some embodiments, the DNA vaccine and the antigen peptide vaccine are co-formulated in a vaccine formulation or each formulated as a separate vaccine formulation, with a pharmaceutically acceptable vehicle.

In some embodiments, the DNA vaccine and the antigen peptide vaccine are formulated as a vaccine formulation suitable for co-delivery through intramuscular injection.

In another aspect, the present disclosure provides a method for preventing or treating SARS-CoV-2 virus infection in a subject, which comprises administering to the subject an effective amount of a DNA vaccine and an effective amount of an antigen peptide vaccine, wherein the DNA vaccine comprises a polynucleotide sequence encoding a polypeptide of the SARS-CoV-2 virus; and wherein the antigen peptide is an antigen peptide of the SARS-CoV-2 virus.

In some embodiments, the polynucleotide sequence is codon optimized for expression in the subject.

In some embodiments, the polypeptide is the SARS-CoV-2 spike protein or comprises at least a conserved moiety of the SARS-CoV-2 spike protein.

In some embodiments, the polypeptide comprises RBD of the spike protein.

In some embodiments, the subject is a human being.

In some embodiments, the DNA vaccine is a plasmid constructed from plasmid pSW3891.

In some embodiments, the polynucleotide sequence comprises a sequence as set forth in SEQ ID NO: 3 or 4.

In some embodiments, the antigen peptide comprises at least a conserved moiety of the SARS-CoV-2 spike protein.

In some embodiments, the antigen peptide comprises RBD of the spike protein.

In some embodiments, the antigen peptide is the S1 subunit of the spike protein.

In some embodiments, the antigen peptide comprises an amino acid sequence of SEQ ID NO: 7 or a functional variant with sequence identity of 80% or more to SEQ ID NO: 7.

In some embodiments, the DNA vaccine and the antigen peptide vaccine are co-formulated in a vaccine formulation or each formulated as a separate vaccine formulation, with a pharmaceutically acceptable vehicle.

In some embodiments, the DNA vaccine and the antigen peptide vaccine are co-administrated to the subject.

In some embodiments, the DNA vaccine and the antigen peptide vaccine are co-administrated to the subject at least 3 times.

In some embodiments, the DNA vaccine and the antigen peptide vaccine are administrated through intramuscular injection.

In another aspect, the present disclosure provides a vaccine kit, which comprises a container, the DNA vaccine or the vaccine combination described above within the container, and a label on or associated with the container that indicates that the DNA vaccine or the vaccine combination is for use in preventing or treating SARS-CoV-2 virus infection.

In another aspect, the present disclosure provides uses of the DNA vaccine or the vaccine combination described above in the preparation of a medicament for preventing or treating SARS-CoV-2 virus infection.

In another aspect, the present disclosure provides a medicament for use in preventing or treating SARS-CoV-2 virus infection, which comprises the DNA vaccine or the vaccine combination described above.

The combination of a DNA vaccine encoding the S protein and an antigen vaccine comprising the S1 subunit is able to confer a full protection against the SARS-CoV-2 virus infection.

### BRIEF DESCRIPTION OF THE DRAWINGS

FIG. 1(A) Designs of SARS-CoV-2 spike protein DNA and protein vaccines. In addition to the wild type S gene insert (wt), two versions of codon optimized (opt) S DNA vaccines were produced: full length S insert (FL) and truncated S insert without transmembrane and intracellular components (dTM). For the expression of recombinant S1 protein, the signal peptide of tissue plasminogen activator (tPA) replaced the nature S protein signal peptide (SP). (B) Western blot analysis to examine the expression of S DNA vaccines and recombinant S1 protein vaccine. 293T cells were transiently transfected with either S-FL-opt or S-dTM-opt DNA plasmids and either culture supernatant (Sup) or cell lysate (lysate) were harvested 72 hours later. Recombinant S1 protein was produced from Expi293 cells and purified by HisTrap HP. S1 specific rabbit polyclonal serum L295-IV was used as the detecting antibody.

FIG. 2 Pilot Immunogenicity study of codon optimized and wild type S-expressing DNA vaccines. Individual mouse (A-B, N=6 per group) or monkey (C-F, N=4 per group) received three DNA immunizations as indicated by arrows using the gene gun delivery approach. Mock group received empty DNA vaccine vector as the negative control. ELISA titers are shown as the average OD of each group (A, C) or end titration titers at the peak level Day 42 (D). Neutralizing antibody responses (NAb) (C) or T cell responses (E and F) are shown from each animal at the peak level Day 42.

FIG. 3 Relative immunogenicity studies in NZW rabbits. Animals were immunized three times at Weeks 0, 2 and 8 by intramuscular needle inoculations. Peak level (2 weeks after the last immunization) S-specific IgG titers (A & C) and NAb responses (B & D) were measured either among codon optimized DNA alone and DNA prime-protein boost approaches (A & B) or among DNA alone, protein alone, DNA prime-protein boost and co-delivery of DNA and protein approaches (C & D).

FIG. 4 Non-human primate immunogenicity and protection study. Animals were immunized three times at Weeks 0, 2 and 8 by intramuscular needle inoculations. Peak level (2 weeks after the last immunization) S-specific IgG titers (A), NAb responses (B) and S-specific IFN-g (C) and Specific-IL-4 (D) responses were measured.

FIG. 5 Viral RNA load detected at various NHP tissues after challenge. Monkeys immunized with various vaccine approaches as described in FIG. 4 were challenged with live SARS-CoV-2 virus through tracheal route and animals were sacrificed 7 days later and viral load (copies/ug) was measured.

FIG. 6 Histology analysis of key organ tissue samples including lung (A) and trachea (B). Mock, protein alone, DNA alone or co-delivery of DNA and protein vaccine.

FIG. 7 Plasmid map of pCW1093 with an S-dTM-opt insert. Pcmv IE: CMV immediate early promoter, sequence location:103-690; Intron A: CMV intron A fragment, sequence location: 825-1650; S: SARS-CoV2 S protein full length coding gene, sequence location: 1678-5499; bGH polyA: bovine growth hormone gene polyadenylation signal, sequence location: 5634-5858; pMB1 ori: pMB1 plasmid replicon, sequence location: 6736-7464; Kanr: aminoglycoside phosphotransferase gene, sequence location:7566-8381.

### DETAILED DESCRIPTION

Unless defined otherwise, technical and scientific terms used herein have the same meaning as commonly understood by a person of ordinary skill in the art. Any methods, devices and materials similar or equivalent to those described herein can be used in the practice of the present invention. The following definitions are provided to facilitate understanding of certain terms used herein and are not meant to limit the scope of the present disclosure.

The articles “a” and “an” are used herein to refer to one or to more than one (i.e., to at least one) of the grammatical object of the article. By way of example, “an element” means one element or more than one element.

As used herein, a “DNA vaccine” refers to a DNA molecule which comprises a DNA sequence encoding a protein antigen, and, after being administrated to a subject (e.g., a human being), leads to humoral (and cell-mediated) immune to the antigen in the subject. Generally, the DNA sequence encoding the protein antigen is operably linked to an expression control sequence, such as a promoter, or array of transcription factor binding sites. The expression control sequence directs transcription of the DNA sequence. The DNA vaccine may include both naked DNA vaccines, e.g., plasmid vaccine, and viral vector-based DNA vaccines that are delivered as viral particles. DNA vaccines afford advantages over conventional vaccines including ease of production, stability, and transport at room temperature.

As used herein, an “antigen peptide vaccine” refers to a protein antigen that will stimulate a host's immune system to make a humoral and/or cellular antigen-specific response. The antigen peptide contains one or more epitopes (either linear, conformational or both). Normally, an epitope will comprise between about 7 and 15 amino acids, such as, 9, 10, 12 or 15 amino acids. An antigen peptide can be obtained by various methods known in the art.

The term “expression” refers to the biological production of a product encoded by a coding sequence. In most cases, the coding sequence, is transcribed to form a messenger-RNA (mRNA). The messenger-RNA is then translated to form a polypeptide product which has a relevant biological activity. Also, the process of expression may involve further processing steps to the RNA product of transcription, such as splicing to remove introns, and/or post-translational processing of a polypeptide product.

“Codon optimization” or “codon optimized for expression” refers to modifying a DNA sequence for enhanced expression in the cells of a subject of interest, e.g., human, by replacing at least one, more than one, or a significant number, of codons of the native sequence with codons that are more frequently or most frequently used in the genes of that subject.

A “conserved moiety” of a protein refers to a protein fragment that is conserved across a protein that may have high sequence diversity in nature, e.g., a viral protein. The conserved moiety needs not have 100% sequence identity across the diversity of naturally occurring sequence of the protein, but the sequence variability in the naturally occurring sequences is low, e.g., less than 10% or 5%.

A “functional variant” of an amino acid sequence refers to any variant exhibiting one or more functional properties identical or similar to those of the amino acid sequence with which it is compared, e.g., it is a functional equivalent. With respect to an antigen peptide, one particular function is the ability to elicit the production of neutralizing antibodies against a virus, when administered to a mammalian subject. In some embodiments, such functional variants may have at least about 50, 55, 60, 65, 70, 75, 80, 85, 90, 95, 96, 97, 98, 99% or more of the activity of the antigen peptides with a sequence as set forth in SEQ ID NO: 7, when measured using standard tests recognized by those of skill in the art. Functional variants may include peptides which have changes or mutations (e.g., at least about one, two, or four, and/or generally less than 15, 10, 5, or 3) relative to the sequence described herein (e.g., conservative or non-essential amino acid substitutions), which do not have a substantial effect on peptide function. Whether or not a particular substitution will be tolerated, i.e., will not adversely affect biological properties, can be predicted, e.g., by evaluating whether the mutation is conservative.

“Vaccine combination” refers to a combination of a DNA vaccine and an antigen peptide vaccine which are administrated to the same subject to elicit an immune response. In some embodiments, the DNA vaccine and the antigen peptide vaccine are co-formulated in a single vaccine formulation with a pharmaceutically acceptable vehicle. In other embodiments, the DNA vaccine and the antigen peptide vaccine are each formulated as a separate vaccine formulation with a pharmaceutically acceptable vehicle.

In embodiments where the DNA vaccine and the antigen peptide vaccine are separate vaccine formulations, the DNA vaccine and the antigen peptide vaccine can be administrated to the subject simultaneously (i.e., co-administrated) or sequentially.

As used herein, “administrated simultaneously”, “co-administrated” or “co-delivered” means that the DNA vaccine and the antigen peptide vaccine are administrated to the same subject at the same time or at substantially the same time. For example, the DNA vaccine is administrated firstly, and, within 1 hour, 1 day, or 2 days, the antigen peptide vaccine is administrated. In these cases, the antigen peptide vaccine is often administrated before the firstly administrated DNA vaccine is able to induce an effective immune response in the subject. This administration scheme is therefore different from the “prime-boost” approach. By contrast, “administrated sequentially” means two vaccines are administrated at different times in which the response to the first vaccine is boosted by a second vaccine comprising the same or different antigen than the first vaccine.

As used herein, “treatment” or “treating” includes any actions which may lead to any beneficial or desirable effect on the symptoms or pathology of a disease (or disorder) in a subject, even minimal reductions in one or more measurable markers of the disease being treated. “Treating” can optionally involve delaying of the progression of the disease. “Treatment” does not necessarily indicate complete eradication or cure of the disease, or associated symptoms thereof.

As used herein, the term “prevention” or “preventing” involves the implementation of necessary practices to prevent the occurrence of a disease or reduce the possibility of the occurrence of a disease in a subject. It does not imply that the disease will not occur.

As used herein, the term “subject” refers to an organism to which the vaccine(s) of the present invention will be administered. Preferably, a subject is a bird or a mammal, e.g., a human being, primate, livestock animal, or a rodent.

The vaccine(s) of the present invention can be used as pure compound, or can be formulated with a pharmaceutically acceptable carrier to form a vaccine formulation. Pharmaceutically acceptable carriers are well known in the art and include, for example, aqueous solutions such as water or physiologically buffered saline or other solvents. In preferred embodiments, when such pharmaceutical compositions are for human administration, e.g., for parenteral administration, the aqueous solution is pyrogen-free, or substantially pyrogen-free.

The route of administration of the vaccine(s) of the present invention may be by parenteral, subcutaneous, intravenous, intramuscular, intraperitoneal, intraarterial, intralesional, intraarticular, topical, oral, rectal, nasal, or any other suitable route.

An “effective amount” of the vaccine(s) of the present invention for preventing or treating SARS-CoV-2 virus infection may vary according to factors such as the disease state, age, sex, and weight of a subject (e.g., a patient). The precise amount contemplated in particular embodiments, to be administered, can be determined by a physician in view of the condition of the subject.

In the current study, we developed a novel subunit COVID-19 vaccine including the S full length DNA plasmid and S1 recombinant protein co-delivered at the same time. This design is most effective in eliciting the higher immune responses including protective antibody and T cell responses than using either DNA or protein component alone. More significantly, this novel COVID-19 vaccine design was able elicit a full protection against the challenge of SARS-CoV-2 in an NHP model (e.g., Rhesus Macaque) which has not been easy to achieve by previous COVID-19 vaccine studies in similar NHP models.

Some embodiments of the present invention are at least partially based on the surprising findings that, the wild type DNA sequences encoding the S protein do not elicit an S-specific antibody responses, while codon optimized DNA sequences encoding the same protein (or its truncated soluble form) are able to elicit a significant S-specific antibody response. Some embodiments of the present invention are at least partially based on the surprising findings that, a combination of a DNA vaccine encoding the S protein and an antigen vaccine comprising the S1 subunit is able to confer a full protection against the SARS-CoV-2 virus infection in NHP model studies.

### DNA Vaccine Construction and Production

The wildtype (S-FL-wt) and codon optimized SARS-CoV-2 spike protein (SEQ ID NO: 1) full length gene sequence (S-FL-opt) were commercially synthesized based on the Wuhan-Hu-1 (GenBank:MN908947). The soluble S ectodomain insert sequence (S-dTM-opt) was generated from the full length opt sequence using the oligomers w1404-TACCGAGCTCGGATCCGCCACCAT (SEQ ID NO: 12) and w1406-GATATCTGCAGAATTCTCAAGGCCACTTGATGTACTGCTCG (SEQ ID NO: 13). All three inserts (S-FL-wt (SEQ ID NO: 2), S-FL-opt (SEQ ID NO: 3) and S-dTM-opt (SEQ ID NO: 4)) were individually subcloned into the mammalian expression plasmid pcDNA3.1+ between BamHI and EcoRI by In-Fusion cloning technology (TAKARA Bio, China). These S-expressing DNA vaccine plasmids were purified from E. coli (TAKARA Bio, China) using endotoxin-free plasmid Maxi kit (Qiagen, USA). All plasmid sequences were confirmed by Sanger DNA sequencing.

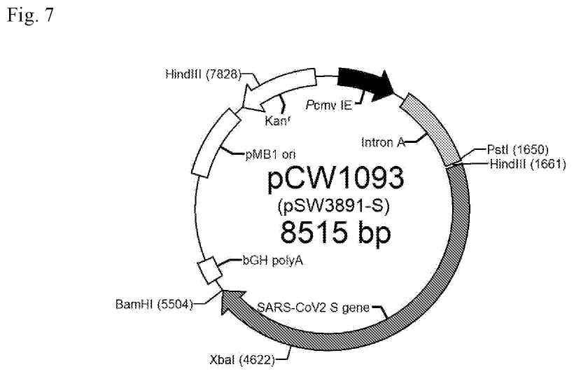

The DNA vaccine pCW1093 was produced by subcloning the S-FL-opt insert into the DNA vaccine vector pSW3891 which can be used in humans as previously reported (34). The insert was amplified from the S-FL-opt template by using the oligomers w1477-TCCATGGGTCTTTTCTGCAGTCACCGTCCAAGCTTGCAATCGCCACCATGTT CGTGTTCCT (SEQ ID NO: 5) and w1479-GGGATTGCGAGGATCCTTATCATGTGTAGTGGAGCTTCACG (SEQ ID NO: 6) and fused into linearized pSW3891 at PstI and BamHI sites. The pCW1093 plasmid (FIG. 7, SEQ ID NO: 11) was transformed into competent E. coli (Thermo Fisher Scientific, USA), single clones were picked up and amplified to produce the final master seed lot (MSL) and working seed lot (WSL). The pCW1093 DNA plasmid used in the non-human primate challenge study was produced under conditions required by the current good manufacturing practices (cGMP) regulation. Bacteria from WSL were gradually expanded to the fermenter and the pCW1093 DNA plasmids were released from final fermentation bacteria pellet by alkaline lysis. The supercoil plasmid DNA is further purified by filtration, chromatography and ultrafiltration. Supercoil plasmid DNA were tested and buffered by saline solution for immunization use.

### S1 Protein Production and Use

Codon optimized version of gene sequence encoding for S1 protein (SEQ ID NO: 7) was subcloned into the mammalian expression vector for in vitro production of recombinant S1 protein for research study applications, and a His-tag was added to the C-terminal of S1 protein for purification purpose. The Expi293 cells (Invitrogen, US) were transfected with the S1-expressing plasmid, the supernatant of cell culture was harvested on Day 5 and the S1 protein was purified by HisTrap HP column. The quality was verified by SDS-PAGE and Western blot analysis before being used for immunization and ELISA study purposes. For immunization, S1 protein was absorbed with aluminum hydroxide (Brenntag Biosector, Frederikssund, Denmark) at a ratio of 1:3.5 (w/w).

### Western Blot Analysis

S-expressing DNA vaccines were tested for their in vitro expression in transiently transfected 293T cells using PEI as the transfecting agent as previously reported (35). 72 hours after the transfection, culture supernatants or cell lysates were subject to Western blot analysis with a rabbit polyclonal serum L295-IV specific for S protein of SARS-CoV-2 virus as the detecting antibody. Similarly, recombinant S1 protein purified from Expi293 cell production was tested with western blot using the same rabbit polyclonal serum.

### Animal Immunizations

### Pilot Animal Studies

Pilot animal DNA immunization studies were conducted in mice and non-human primates to compare the relative immunogenicity of different S-expressing DNA vaccine constructs (S-FL-wt, S-FL-opt and S-dTM-opt). Either 6-8 weeks old C57BL/6N mice or 3-4 years old rhesus monkeys were immunized three time at Weeks 0, 2 and 4 with 5 μg DNA each time delivered by a Helio Gene Gun (Bio-Rad, USA). Serum samples were collected prior to the start of the study or 14 days after each immunization.

Additional pilot study was conducted in New Zealand White (NZW) rabbits. Rabbits were either immunized with DNA vaccines (S-FL-opt) three times (Weeks 0, 2 and 6) with 200 μg DNA vaccine each time by needle intramuscular injection (IM), or received twice IM DNA immunizations (S-FL-opt or S-dTM-opt) at Weeks 0 and 2, followed by one time IM injection of 50 μg recombinant S1 protein vaccine at Week 6. Serum samples were collected prior to the start of the study or 14 days after the 3^{rd} immunization.

### Optimal Vaccination Design Studies

The relative immunogenicity of different vaccination designs (S-FL-opt DNA alone, S1 protein alone or co-delivery of S-FL-opt DNA+S1 protein vaccines) was further studied in NZW rabbit. All animals received three intramuscular (IM) needle immunizations at Week 0, 2 and 6 with fixed dosing: 200 μg DNA vaccine and 50 μg protein vaccine, delivered either alone or in combination. Serum samples were collected prior to the start of the study or 14 days after the 3^{rd} immunization.

### Non-Human Primate (NHP) Immunogenicity and Protection Study

Groups of randomly assigned 3-4 years old rhesus monkeys were immunized three times at Weeks 0, 2 and 8 with one of the following vaccination regimen: DNA vaccine pCW1093 (2 mg each time), recombinant 51 protein (100 μg each time) or co-delivery of DNA vaccine pCW1093 (2 mg) and 51 protein (100 μg) each time, all delivered by intramuscular needle injections. The control animals received saline injections. Peripheral blood was collected prior to the start of study and 7 days after each immunization for routine blood biochemical tests and SARS-CoV-2 specific immune responses.

A challenge study was conducted at 4 weeks after the third immunization by direct inoculation of 5×10⁶ TCID50 of SARS-CoV-2 virus through the intratracheal route under anesthesia. Throat and anal swabs were collected at 0, 2, 4, 6 and 7 days after challenge and used to determine the viral load. At seven days after challenge, all animals were euthanized, the viral load in the different tissue was detected, and a pathological examination was conducted.

### Virus and Cell Line

SARS-CoV-2 strain BP16 was isolated from the sputum of a COVID-19 patient in Kunming, Yunnan, and amplified in Vero cells. The viral genome was extracted and subjected to nanopore sequencing (Nextomics Bioscience, Wuhan). The BP16 complete genome contains two mutations, C8782T and T28144C aligned with Wuhan-Hu-1. The former is a silent mutation, and the latter resulting in an amino acid difference in the ORF8 (L84S). BP16 was used in the neutralization and challenge assay. Vero cells were used for the production and titration of SARS-CoV-2 stocks. Vero cells were maintained in Dulbecco's modified Eagle's medium (DMEM, Corning) supplemented with 10% fetal bovine serum (FBS, Gibco,) 100 IU/mL penicillin, and 100 μg/mL streptomycin, and incubated at 37° C., 5% CO₂. The SARS-CoV-2 virus titer was determined by a micro-dose cytopathogenic efficiency (CPE) assay. Serial 10-fold dilutions of virus-containing samples were mixed with 2×10⁴ Vero cells and then plated in 96-well culture plates. After 5 days of culture in a 5% CO₂ incubator at 37° C., cells were checked for the presence of a CPE under a microscope. Titers for SARS-CoV-2 were resolved by a 50% tissue-culture infectious doses (TCID50) assay.

### ELISA

The 96-well ELISA plates (Corning, USA) were coated with 0.2 μg/well S1 protein in 100 μL coating buffer (15 mM Na₂CO₃ and 35 mM NaHCO₃, pH 9.6) and incubated at 4° C. overnight. Plates were washed in PBST (0.5% TWEEN-20/PBS) and wells blocked using 2% BSA/PBST for 1 hr at 30° C. Serially diluted serum samples were added and incubated for 1 hr at Plates were washed and horseradish peroxidase-conjugated goat anti-mouse IgG or anti-rabbit IgG (Invitrogen, USA) or horseradish peroxidase-conjugated goat anti-monkey IgG (Santa Cruz Biotechonology, USA) was added to all wells for 1 hr at 30° C. The reaction was developed using TMB substrate (Makewonderbio, Beijing, China) and determined at 450 nm by a microplate reader.

### Neutralization Antibody Assays

Two neutralization assays were used in the current report. The first one was conducted at IMB based on the neutralizing activities against real SASR-CoV-2 virus infection to Vero cells. In this assay, mouse or NHP serum samples collected from immunized animals were heat-inactivated at 56° C. for 30 min and serially diluted with virus dilution medium at a starting dilution of 1:4 and then serially diluted 2-fold up to the required concentration. An equal volume of challenge virus solution containing 100 TCID50 virus was added, followed by 1 hour incubation at 37° C. 1×10⁴ Vero cells were then added to the serum-virus mixture, and the plates were incubated for 5 days at 37° C. in a 5% CO₂ incubator. Cytopathic effect (CPE) of each well was recorded under microscopes, and the neutralizing titer was calculated by the dilution number of 50% protective condition.

The second neutralization assay is a pseudotyped virus based assay conducted at UMMS. The heat-inactivated immune rabbit serum samples were serially diluted at a starting dilution of 1:20 with 2-fold serial dilutions in 55 μl of volume. An equal volume of SARS-CoV-2 pseudovirus (100 TCID₅₀/mL) was added, followed by 1 hour incubation at 37° C. Then take 100 μl of the serum/virus mixture and add it to the 96 well plates proceeded with 1×10⁴ Vero-E6 cells per well. After the plates were incubated for 24 hours at 37° C. with 5% CO₂, 100 μl/well fresh media was fed. At 48 hours after the infection, cells were washed with PBS and then lysed using passive lysis buffer. The luciferase activities developed with Luciferase substrate (Promega) and read. Neutralization was calculated as the percent change in luciferase activity in the presence of preimmune sera versus that of luciferase activity in the presence of immune sera [(Preimmune RLUs−Immune RLUs)/(Preimmune RLUs)]×100. The NAb titers were determined at the serum dilution with 50% neutralization.

### ELISpot Assay

Immunized macaque PBMCs were isolated to evaluate the antigen-specific T cell responses by ELISpot^{PLUS} (ALP) kits (Mabtech, Sweden). The ELISPOT plates were incubated with 200 μl/well of serum-free media for 30 minutes at room temperature. Then add 50 μl/well of pooled peptides (5 μg/peptide/mL) or S1 protein (20 μg/mL) in serum-free media and 50 μl/well of macaque PBMCs at 3×10⁵ cells/well. The plates were incubated for 16 hours at 37° C. with 5% CO₂. After the plates were washed with pre-chilled water and PBS for 5 times, the plates were detected with conjugated anti-cytokine antibodies.

For macaque IFN-γ detections, biotinylated-anti-monkey IFN-γ at 1:1000 dilution in PBS with 0.5% FBS was added at 100 μL/well and incubated for 1 hour at room temperature. Following washes, the plates were further incubated with 100 μl/well of ALP-conjugated-Streptavidin at 1:1000 dilution for 1 hour at room temperature. Following washes with PBS for 5 times, the plates were developed with 100 μl/well of BCIP/NPT-plus substrate for 5 minutes in dark and washed with water and air-dried. For macaque IL-4 detection, the plates were directly incubated with 100 μl/well of ALP-conjugated-anti-human-IL-4 at 1:1000 dilution for 1 hour at room temperature. Following washes with PBS for 5 times, the plates were developed with 100 μl/well of BCIP/NPT-plus substrate for 5 minutes in dark and washed with water and air-dried. The immune spots in the ELISPOT plates were counted using ELISAPOT reader (CTL, USA) and the final sport-forming units (SFUs) were calculated as spots/million cells.

### Realtime-RT-PCR Assay

Tissues were homogenized in TRNzol universal reagent by TGrinder H24 (TIANGEN, China) and RNA was extracted using Direct-Zol RNA Miniprep kit (ZYMO RESEARCH). Viral gRNA was reverse transcribed and amplified by One Step PrimerScript RT-PCR Kit (TakaRa) using Ligtcycler 480II Real-Time PCR System (Roche) according to manufacturer's instructions. Viral loads were calculated as viral RNA copies per mL or per mg tissue and the assay sensitivity was 100 copies. The target for amplification was SARS-CoV2 N (nucleocapsid) gene. The primers and probes for the targets were:

(SEQ ID NO: 8)
N-F: 5′-GGGGAACTTCTCCTGCTAGAAT-3′;
(SEQ ID NO: 9)
N-R: 5′-CAGACATTTTGCTCTCAAGCTG-3′;
(SEQ ID NO: 10)
N-P: 5′-VIC-TTGCTGCTTGACAGATT-BHQ1-3′.

For quantification of viral loads by RT-PCR, A standard curve of Ct values to the copy number of viral RNA is generated with serial 10-fold dilutions of RNA transcribed from recombinant plasmid pcDNA3.1-nCoV N in vitro with a known copy number. The viral loads of each sample were converted with Ct value and the standard curve. Statistical analysis was performed by LightCycler 480 Software.

### Histopathological Analysis

The collected tissue sections (3 mm thickness) were fixed with 4% formaldehyde for 1 week. The fixed tissues were further dehydrated before being sliced into 2-3 μm thickness sections, and flatten on slides in warm water (40° C.). The slides were further dried and dewaxed at 60° C., and were stained with hematoxylin for 3-5 min, differentiated with hydrochloric acid aqueous solution, blue with aqueous ammonia solution, stained with eosin for 5 min after dehydration. The slides were finally sealed with neutral gel.

### Statistical Analyses

Analysis of virologic and immunologic data was performed using GraphPad Prism 8.4.2 (GraphPad Software). Comparison of data between groups was performed using two-sided Mann-Whitney tests. Correlations were assessed by two-sided Spearman rank-correlation tests. Student t-test was used to compare the antibody titers between groups. P-values of less than 0.05 were considered significant.

The current study is designed based on the significant amount of information accumulated in the literature in the last 2 decades including our own work that the immunogenicity of DNA vaccines is limited when used alone (36-38), even with the inclusion of molecular adjuvants such as plasmids expressing immune enhancing cytokines (39, 40). Physical delivery approaches such as gene gun and electroporation can greatly enhance the immunogenicity but the cost and complexity are increased with the use of a physical instrument. One promising option is the heterologous prime-boost or co-delivery of DNA vaccine with another vaccine modality such as recombinant protein vaccines which share the same antigens with the ones expressed by DNA vaccines (41-44).

We have adopted the same concept in the current study to test whether a combination of DNA and protein COVID-19 vaccine can greatly enhance the protective immunogenicity than using either DNA or protein components alone.

### EXAMPLES

### Examples 1

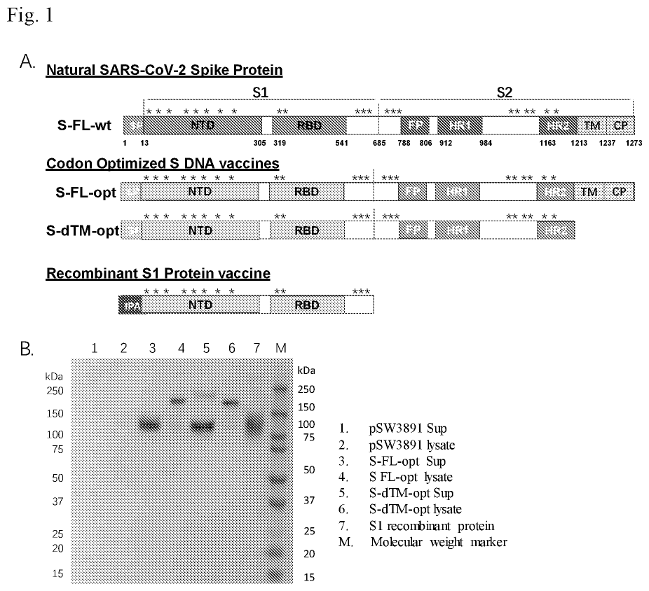

The optimal design of DNA vaccine expressing the S protein of SARS-CoV-2 as the key protective antigen was selected after comparing the immunogenicity of two similar versions of candidate S DNA vaccines. The first one, S-FL-opt, is the full length S gene insert expressing the exact same amino acid sequences as the natural S protein from the SARS-CoV-2 virus (FIG. 1A). The only change is that wild type S gene nucleic acid sequences (-wt) were replaced with the codon optimized S gene sequences (-opt) with the same approach as we previously reported for SARS and influenza DNA vaccines (45, 46). The other S DNA vaccine design included a S-dTM-opt insert which is similar to codon-optimized S-FL-opt but with the truncation of transmembrane and cytoplasmic domains of S protein (FIG. 1A). The expression of S antigens by both DNA vaccine designs was confirmed using in vitro transfection of these DNA plasmids in 293T cells followed by the Western blot analysis (FIG. 1B).

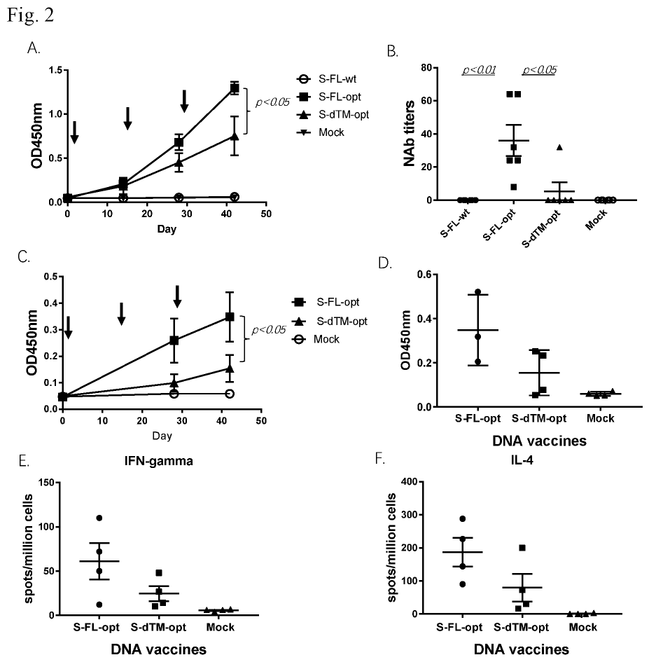

The relative immunogenicity of S-FL-opt and S-dTM-opt DNA vaccines was studied in multiple animal models. First in Balb/C mice using gene gun delivery, both S-FL-opt and S-dTM-opt DNA vaccines elicited S-specific serum antibody responses and the titers went up following each immunization with the same DNA vaccines (FIG. 2A). The peak level antibody responses after three immunizations were statistically different with the titers in the S-FL-opt group being much higher than the S-dTM-opt group. Mice received either the DNA vaccine encoding the wild type gene sequences of full length S gene (S-FL-wt) or the saline (mock) did not have detectable S-specific antibody responses (FIG. 2A). Consistent with the binding antibody data, immune sera from S-FL-opt group had higher neutralizing antibody (NAb) titers than the S-dTM-opt group (p<0.05) and no detectable NAb was detected in either S-FL-wt or mock groups (FIG. 2B). Overall the NAb levels were low in the mouse model when S-expressing DNA vaccines alone were tested.

Then in a pilot non-human primate (NHP) study using gene gun delivery, both the temporal development and the peak level of serum S-specific IgG titers in S-FL-opt group were significantly higher than in S-dTM-opt group (p<0.05) (FIG. 2C-2D). But the NAb responses elicited by either of two S-expression DNA vaccines were low or barely detectable (data now shown). On the other hand, the full length S antigen design (S-FL-opt) was able to induce higher level of IFN-gamma and IL-4 responses than S-dTM-opt group as measured by the ELIspot analysis (FIGS. 2E and 2F).

### Examples 2

With the identification of the most optimal S-expressing DNA vaccine design, a recombinant S1 protein was produced in parallel from transiently transfected Expi293 cells so it can be used to test the DNA and protein combination vaccine strategy. The design of S1 protein gene is shown in FIG. 1A in which a tissue plasminogen activator (tPA) leader replaced the natural signal peptide sequence of S protein from SARS-CoV-2 with the hope to optimize the production of a secreted S1 protein as previously shown with other viral proteins (47). The entire S1 protein sequence including the receptor binding domain (RBD) is preserved as in the original virus. As it is now well known that the production of full length SARS-CoV-2 S recombinant protein is technically challenging as it is unstable and hard to achieve high yield of purified full length recombinant S protein (48-50). Since the RBD is considered as the major target for protective antibody responses, we hypothesized that the S1 protein, instead of the full length S protein, should provide the same boosting effect to focus at the RBD region in a host primed with the full length S DNA vaccine. The recombinant S1 protein used in the current study was partially purified by a research lab based production process as shown in Lane 7, FIG. 1B).

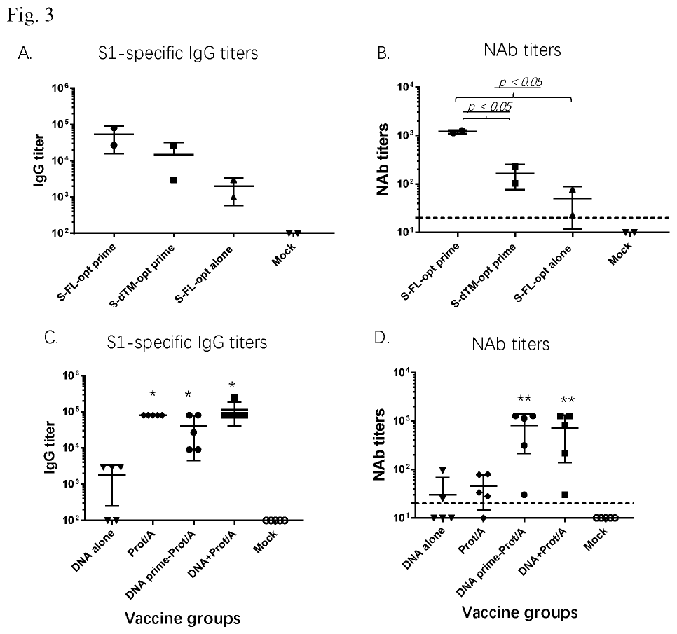

An immunogenicity study was conducted in NZW rabbits to test the immunogenicity of DNA and protein combination vaccine design. Both DNA and protein vaccines in this study were delivered by the traditional needle intramuscular injection (IM). Animals were immunized either with the S-FL-opt DNA vaccine alone, or with a S1 protein boost after priming with one of the two S DNA vaccines (S-FL-opt or S-dTM-opt). The result clearly demonstrated that the protein boost is highly effective in eliciting much higher S-specific IgG responses than giving DNA vaccine alone. The protein boost was able to push the antibody titers in animals primed with the less optimal DNA vaccine S-dTM-opt higher than those only receiving the optimal DNA vaccine S-FL-opt alone. However, after the S1 protein boost, the titers in those primed with the optimal DNA vaccine S-FL-opt were still higher than those primed with the less optimal DNA vaccine S-dTM-opt (FIG. 3A). The prime-boost groups showed easily detectable NAb responses and minimal animal to animal variation within the same group. The S-FL-opt prime+S1 protein boost had the highest titers of NAb (FIG. 3B). These data indicated that priming with the optimal DNA vaccine design is critical, especially to the induction of high level NAb, and the protein boost can further maximize the level of protective antibody responses.

### Examples 3

We next tested the relative immunogenicity between the sequential and the co-delivery of full length S-expressing DNA and S1 protein vaccines in the NZW rabbit model. The co-delivery immunization schedule is reported to be highly immunogenic (51) and is easier to implement in large human populations without tracking when a DNA or a protein vaccine component should be administered as in a sequential prime-boost design. Rabbits receiving the co-delivery of DNA and protein vaccines were able to induce much higher S-specific IgG responses than the DNA alone group, but only slightly better than the protein alone group (FIG. 3C). However, it is very interesting to discover that serum NAb titers in co-delivery group were much higher than both DNA alone or protein alone groups (FIG. 3D). This finding supports the value of DNA vaccines in eliciting highly conformational antibody responses which are critical for protective functions (52, 53). Furthermore, the sequential DNA prime-protein boost approach was slightly less immunogenic in eliciting S1-specific IgG antibody responses than the co-delivery (FIG. 3C) but the NAb responses were very similar between sequential and co-delivery of DNA and protein vaccines (FIG. 3D).

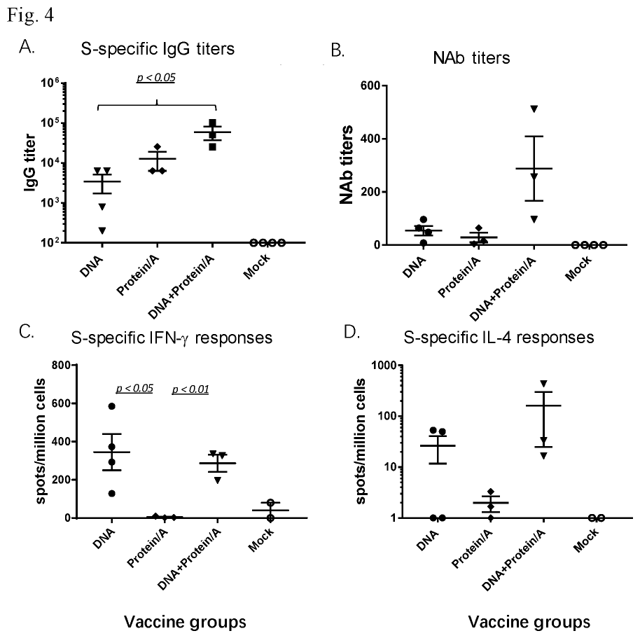

Based on the results of from the above pilot animal studies, the co-delivery of DNA and protein vaccines approach was selected as the leading immunization design of our candidate COVID-19 vaccines and further tested in an NHP protection study against live SARS-CoV-2 viral challenge. As seen in the preliminary rabbit study, co-delivery of S-FL-opt DNA vaccine and recombinant S1 protein vaccine was the most immunogenic design to elicit higher S-specific IgG responses than DNA alone or protein alone groups (p<0.05 in both cases) (FIG. 4A). More significantly, the co-delivery group elicited the highest NAb activities among three vaccine groups (FIG. 4B). Regarding to the T cell immune responses, either DNA alone group or co-delivery group, was able to elicit robust IFN-gamma and IL-4 responses, and such responses were much higher than those detected in protein vaccine alone group (FIG. 4C and FIG. 4D). Our data validated the long time concept that DNA vaccines are good in eliciting T cell immunity (54-56).

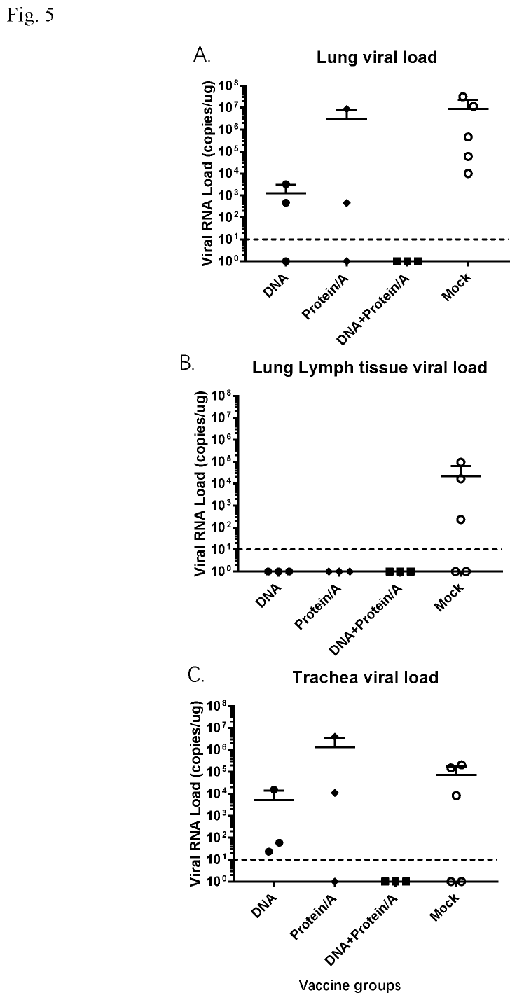

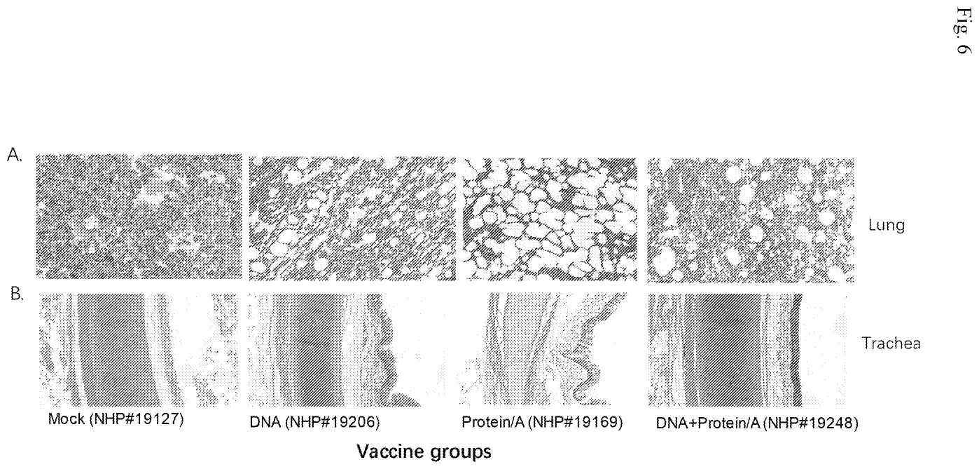

Animals in this NHP study were further challenged with the live SARS-CoV-2 virus through the intratracheal route. The co-delivery of S-FL-opt DNA and recombinant S1 protein vaccines achieved the full protection. No virus was detected in trachea, lung lymph tissues and lung tissues in this group of animals (FIG. 5A-C). For animals in mock group, high levels of viruses were detected in all three tissues. Animals receiving DNA vaccine alone had lower level of viral detection than the mock group but still had viral RNA detection in trachea in three animals and in lung in two animals (FIG. 5A-C). Histology analysis of sacrificed animal lung tissue showed severe inflammation in mock monkey lung samples while any of the COVID-19 vaccine approach was able to greatly reduce the inflammation (FIG. 6A). Similarly, the mucosal surface was severely damaged in mock group monkey but not in monkeys included in any of the COVID-19 vaccination groups (FIG. 6B). Combining the better immunogenicity of antibody and T cell responses and the full protection against viral challenge, our data strongly support the development of a potent COVID-19 vaccine based on the novel DNA and protein combination formulation which may provide strong and long lasting immune protection.

### Discussion

A safe and efficacious SARS-CoV-2 vaccine is needed to end the global COVID-19 pandemic. Multiple vaccine candidates have advanced to Phase III human efficacy trials with some of them are expected to receive regulatory approval in near future for possible use in certain high risk populations. However, very little is currently known about the real protection efficacy of these leading vaccine candidates and more importantly how long the immune protection may last with these candidates. It is prudent to develop the next generation of vaccines which will be able to elicit stronger immune responses and better protection against SARS-CoV-2 viral infection than the first generation of COVID-19 vaccines under development.

In this study, we analyzed the relative immunogenicity of DNA, protein and the combination of DNA and protein vaccines. We demonstrated while codon optimization and optimal S gene insert design may produce the more immunogenic S-expressing DNA vaccines as previously reported (57), the combination of DNA and protein together can significantly improve the overall anti-S antibody responses and specifically the NAb responses than the DNA or protein vaccines alone approaches. Both the sequential DNA prime-protein boost and co-delivery of DNA and protein components at the same time are similarly effective in eliciting high level protective antibody responses, indicating the value of DNA vaccine in generating antibodies against conformation sensitive epitopes (52, 53). Co-delivery approach may be more practical for large scale human population applications as the vaccine formulation will be same for each time of immunization and no need to worry whether a DNA or protein vaccine should be administered as in the sequential prime and boost regimen.

By using the co-delivery of DNA and protein vaccines approach, we demonstrated that it can elicit full protection in all monkeys receiving such a combination vaccine formulation without any detectable viruses in all studied tissues compared with sham control animals. Both DNA vaccine alone or protein vaccine alone approaches achieved viral load reduction in various tissues as reported by other current COVID-19 vaccines but not full protection in any of the immunized monkeys in these two groups.

Our data extend previous studies showing the DNA and protein combination vaccines are more effective than either component alone in eliciting potent immune responses against HIV-1 or influenza (36, 37, 42). DNA immunization can use both innate immunity and acquired immunity mechanisms as we reported (58-61) to induce the development of antigen specific B cell responses especially those germinal center B cells which is the basis for much amplified antibody responses upon the boost of a protein vaccine. It is now known that SARS-CoV-2 infection does not establish long lasting antibody responses in patients who had mild clinical symptoms indicting the potential low immunogenicity of its S antigen. Such findings imply that a successful COVID-19 vaccine needs to elicit stronger than the natural infection, and a long-lasting immune responses including a long-lasting S-specific memory B cells may be critical. Our approach including the DNA component will greatly facilitate this process. More significantly, the inclusion of a DNA vaccine component can serve two important purposes: 1) to improve the quality of antibody responses such as the levels of NAb, due the ability of DNA vaccines to induce better antibody responses against conformational epitopes (52, 53) and 2) to elicit high levels of antigen specific memory B cells through better activation of germinal center B cell development than protein based vaccines (60, 61).

However, as shown in this study again, the immunogenicity of even optimized DNA vaccine still has its limits on how high the antibody responses may be elicited, and the addition of a protein vaccine can further push the limit higher. Low immunogenicity is a common feature for all kinds of nucleic acid vaccines including both DNA and RNA vaccines when used alone. As we learned in the last two decades, strategies such as enhanced delivery, using immune stimulating cytokines or adjuvants, and physical delivery tools (gene gun or electroporation) can only partially improve the immunogenicity of nucleic acid based vaccines (62) and may also bring additional issues such as safety, cost and complexity of use. The combination DNA and protein vaccine strategy offers a unique solution to maximize the efficacy of two vaccine modalities without causing any additional safety concern (41, 63). While we focused on the prime-boost approach in the past, our current data proved that co-delivery of DNA and protein vaccines at the same time could also produce higher immune responses and enhanced vaccine protection against SARS-CoV-2 in a non-human primate model The DNA and protein combination formulation should be considered as a leading candidate for the next generation of improved COVID-19 vaccines if a high immune responses and long lasting immunity are needed to achieve the full control of COVID-19 from a global scale.

Listed below are some amino acid sequences and nucleic acid sequences mentioned herein.

amino acid sequence of S protein
(SEQ ID NO: 1)
MFVFLVLLPLVSSQCVNLTTRTQLPPAYTNSFTRGVYYPDKVFRSSVLHSTQDLFL
PFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTITDAV
DCALDPLSETKCTLKSFTVEKGIYQTSNFRVQPTESIVRFPNITNLCPFGEVENATRFAS
VYAWNRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKSNLKPF
ERDISTEIYQAGSTPCNGVEGENCYFPLQSYGFQPTNGVGYQPYRVVVLSFELLHAPAT
VCGPKKSTNLVKNKCVNFNFNGLTGTGVLTESNKKFLPFQQFGRDIADTTDAVRDPQ
TLEILDITPCSFGGVSVITPGTNTSNQVAVLYQDVNCTEVPVAIHADQLTPTWRVYSTG
SNVFQTRAGCLIGAEHVNNSYECDIPIGAGICASYQTQTNSPRRARSVASQSIIAYTMSL
GAENSVAYSNNSIAIPTNFTISVTTEILPVSMTKTSVDCTMYICGDSTECSNLLLQYGSF
CTQLNRALTGIAVEQDKNTQEVFAQVKQIYKTPPIKDFGGFNFSQILPDPSKPSKRSFIE
DLLFNKVTLADAGFIKQYGDCLGDIAARDLICAQKFNGLTVLPPLLTDEMIAQYTSAL
LAGTITSGWTFGAGAALQIPFAMQMAYRENGIGVTQNVLYENQKLIANQFNSAIGKIQ
DSLSSTASALGKLQDVVNQNAQALNTLVKQLSSNFGAISSVLNDILSRLDKVEAEVQI
DRLITGRLQSLQTYVTQQLIRAAEIRASANLAATKMSECVLGQSKRVDFCGKGYHLMS
FPQSAPHGVVFLHVTYVPAQEKNFTTAPAICHDGKAHFPREGVFVSNGTHWFVTQRN
FYEPQIITTDNTFVSGNCDVVIGIVNNTVYDPLQPELDSFKEELDKYFKNHTSPDVDLG
DISGINASVVNIQKEIDRLNEVAKNLNESLIDLQELGKYEQYIKWPWYIWLGFIAGLIAI
VMVTIMLCCMTSCCSCLKGCCSCGSCCKFDEDDSEPVLKGVKLHYT.
S protein coding sequence
(S-FL-wt, SEQ ID NO: 2)
ATGTTTGTTTTTCTTGTTTTATTGCCACTAGTCTCTAGTCAGTGTGTTAATCTTA
CAACCAGAACTCAATTACCCCCTGCATACACTAATTCTTTCACACGTGGTGTTTATT
ACCCTGACAAAGTTTTCAGATCCTCAGTTTTACATTCAACTCAGGACTTGTTCTTAC
CTTTCTTTTCCAATGTTACTTGGTTCCATGCTATACATGTCTCTGGGACCAATGGTA
CTAAGAGGTTTGATAACCCTGTCCTACCATTTAATGATGGTGTTTATTTTGCTTCCA
CTGAGAAGTCTAACATAATAAGAGGCTGGATTTTTGGTACTACTTTAGATTCGAAG
ACCCAGTCCCTACTTATTGTTAATAACGCTACTAATGTTGTTATTAAAGTCTGTGAA
TTTCAATTTTGTAATGATCCATTTTTGGGTGTTTATTACCACAAAAACAACAAAAG
TTGGATGGAAAGTGAGTTCAGAGTTTATTCTAGTGCGAATAATTGCACTTTTGAAT
ATGTCTCTCAGCCTTTTCTTATGGACCTTGAAGGAAAACAGGGTAATTTCAAAAAT
CTTAGGGAATTTGTGTTTAAGAATATTGATGGTTATTTTAAAATATATTCTAAGCA
CACGCCTATTAATTTAGTGCGTGATCTCCCTCAGGGTTTTTCGGCTTTAGAACCATT
GGTAGATTTGCCAATAGGTATTAACATCACTAGGTTTCAAACTTTACTTGCTTTAC
ATAGAAGTTATTTGACTCCTGGTGATTCTTCTTCAGGTTGGACAGCTGGTGCTGCA
GCTTATTATGTGGGTTATCTTCAACCTAGGACTTTTCTATTAAAATATAATGAAAAT
GGAACCATTACAGATGCTGTAGACTGTGCACTTGACCCTCTCTCAGAAACAAAGTG
TACGTTGAAATCCTTCACTGTAGAAAAAGGAATCTATCAAACTTCTAACTTTAGAG
TCCAACCAACAGAATCTATTGTTAGATTTCCTAATATTACAAACTTGTGCCCTTTTG
GTGAAGTTTTTAACGCCACCAGATTTGCATCTGTTTATGCTTGGAACAGGAAGAGA
ATCAGCAACTGTGTTGCTGATTATTCTGTCCTATATAATTCCGCATCATTTTCCACT
TTTAAGTGTTATGGAGTGTCTCCTACTAAATTAAATGATCTCTGCTTTACTAATGTC
TATGCAGATTCATTTGTAATTAGAGGTGATGAAGTCAGACAAATCGCTCCAGGGC
AAACTGGAAAGATTGCTGATTATAATTATAAATTACCAGATGATTTTACAGGCTGC
GTTATAGCTTGGAATTCTAACAATCTTGATTCTAAGGTTGGTGGTAATTATAATTA
CCTGTATAGATTGTTTAGGAAGTCTAATCTCAAACCTTTTGAGAGAGATATTTCAA
CTGAAATCTATCAGGCCGGTAGCACACCTTGTAATGGTGTTGAAGGTTTTAATTGT
TACTTTCCTTTACAATCATATGGTTTCCAACCCACTAATGGTGTTGGTTACCAACCA
TACAGAGTAGTAGTACTTTCTTTTGAACTTCTACATGCACCAGCAACTGTTTGTGG
ACCTAAAAAGTCTACTAATTTGGTTAAAAACAAATGTGTCAATTTCAACTTCAATG
GTTTAACAGGCACAGGTGTTCTTACTGAGTCTAACAAAAAGTTTCTGCCTTTCCAA
CAATTTGGCAGAGACATTGCTGACACTACTGATGCTGTCCGTGATCCACAGACACT
TGAGATTCTTGACATTACACCATGTTCTTTTGGTGGTGTCAGTGTTATAACACCAG
GAACAAATACTTCTAACCAGGTTGCTGTTCTTTATCAGGATGTTAACTGCACAGAA
GTCCCTGTTGCTATTCATGCAGATCAACTTACTCCTACTTGGCGTGTTTATTCTACA
GGTTCTAATGTTTTTCAAACACGTGCAGGCTGTTTAATAGGGGCTGAACATGTCAA
CAACTCATATGAGTGTGACATACCCATTGGTGCAGGTATATGCGCTAGTTATCAGA
CTCAGACTAATTCTCCTCGGCGGGCACGTAGTGTAGCTAGTCAATCCATCATTGCC
TACACTATGTCACTTGGTGCAGAAAATTCAGTTGCTTACTCTAATAACTCTATTGCC
ATACCCACAAATTTTACTATTAGTGTTACCACAGAAATTCTACCAGTGTCTATGAC
CAAGACATCAGTAGATTGTACAATGTACATTTGTGGTGATTCAACTGAATGCAGCA
ATCTTTTGTTGCAATATGGCAGTTTTTGTACACAATTAAACCGTGCTTTAACTGGAA
TAGCTGTTGAACAAGACAAAAACACCCAAGAAGTTTTTGCACAAGTCAAACAAAT
TTACAAAACACCACCAATTAAAGATTTTGGTGGTTTTAATTTTTCACAAATATTAC
CAGATCCATCAAAACCAAGCAAGAGGTCATTTATTGAAGATCTACTTTTCAACAAA
GTGACACTTGCAGATGCTGGCTTCATCAAACAATATGGTGATTGCCTTGGTGATAT
TGCTGCTAGAGACCTCATTTGTGCACAAAAGTTTAACGGCCTTACTGTTTTGCCAC
CTTTGCTCACAGATGAAATGATTGCTCAATACACTTCTGCACTGTTAGCGGGTACA
ATCACTTCTGGTTGGACCTTTGGTGCAGGTGCTGCATTACAAATACCATTTGCTAT
GCAAATGGCTTATAGGTTTAATGGTATTGGAGTTACACAGAATGTTCTCTATGAGA
ACCAAAAATTGATTGCCAACCAATTTAATAGTGCTATTGGCAAAATTCAAGACTCA
CTTTCTTCCACAGCAAGTGCACTTGGAAAACTTCAAGATGTGGTCAACCAAAATGC
ACAAGCTTTAAACACGCTTGTTAAACAACTTAGCTCCAATTTTGGTGCAATTTCAA
GTGTTTTAAATGATATCCTTTCACGTCTTGACAAAGTTGAGGCTGAAGTGCAAATT
GATAGGTTGATCACAGGCAGACTTCAAAGTTTGCAGACATATGTGACTCAACAATT
AATTAGAGCTGCAGAAATCAGAGCTTCTGCTAATCTTGCTGCTACTAAAATGTCAG
AGTGTGTACTTGGACAATCAAAAAGAGTTGATTTTTGTGGAAAGGGCTATCATCTT
ATGTCCTTCCCTCAGTCAGCACCTCATGGTGTAGTCTTCTTGCATGTGACTTATGTC
CCTGCACAAGAAAAGAACTTCACAACTGCTCCTGCCATTTGTCATGATGGAAAAG
CACACTTTCCTCGTGAAGGTGTCTTTGTTTCAAATGGCACACACTGGTTTGTAACA
CAAAGGAATTTTTATGAACCACAAATCATTACTACAGACAACACATTTGTGTCTGG
TAACTGTGATGTTGTAATAGGAATTGTCAACAACACAGTTTATGATCCTTTGCAAC
CTGAATTAGACTCATTCAAGGAGGAGTTAGATAAATATTTTAAGAATCATACATCA
CCAGATGTTGATTTAGGTGACATCTCTGGCATTAATGCTTCAGTTGTAAACATTCA
AAAAGAAATTGACCGCCTCAATGAGGTTGCCAAGAATTTAAATGAATCTCTCATC
GATCTCCAAGAACTTGGAAAGTATGAGCAGTATATAAAATGGCCATGGTACATTT
GGCTAGGTTTTATAGCTGGCTTGATTGCCATAGTAATGGTGACAATTATGCTTTGC
TGTATGACCAGTTGCTGTAGTTGTCTCAAGGGCTGTTGTTCTTGTGGATCCTGCTGC
AAATTTGATGAAGACGACTCTGAGCCAGTGCTCAAAGGAGTCAAATTACATTACA
CATAA
S protein coding sequence
(S-FL-opt, SEQ ID NO: 3)
ATGTTCGTGTTCCTGGTGCTCCTCCCTCTCGTGTCTTCTCAGTGCGTGAACCTG
ACCACACGGACCCAGCTGCCACCCGCTTACACCAACTCCTTCACAAGAGGCGTGT
ACTACCCCGACAAGGTGTTCCGGTCTTCTGTGCTCCACTCTACCCAGGACCTGTTC
CTGCCATTCTTCTCTAACGTGACATGGTTCCACGCTATCCACGTGTCTGGCACCAA
CGGCACAAAGAGATTCGACAACCCCGTGCTCCCATTCAACGACGGCGTGTACTTC
GCTAGCACAGAGAAGTCCAACATCATCCGGGGCTGGATCTTCGGCACCACACTGG
ACTCTAAGACCCAGTCCCTCCTCATCGTGAACAACGCCACAAACGTGGTGATCAA
GGTGTGCGAGTTCCAGTTCTGCAACGACCCATTCCTGGGCGTGTACTACCACAAGA
ACAACAAGTCTTGGATGGAGAGCGAGTTCCGGGTGTACTCTAGCGCTAACAACTG
CACATTCGAGTACGTGTCTCAGCCATTCCTCATGGACCTCGAGGGCAAGCAGGGC
AACTTCAAGAACCTGAGGGAGTTCGTGTTCAAGAACATCGACGGCTACTTCAAGA
TCTACTCTAAGCACACCCCAATCAACCTGGTGAGGGACCTGCCTCAGGGCTTCTCC
GCTCTCGAGCCACTGGTGGACCTGCCAATCGGCATCAACATCACCCGGTTCCAGAC
ACTCCTCGCCCTCCACCGGTCTTACCTGACCCCAGGCGACTCTAGCAGCGGCTGGA
CCGCCGGCGCCGCCGCTTACTACGTGGGCTACCTGCAGCCTAGGACATTCCTCCTG
AAGTACAACGAGAACGGCACAATCACCGACGCTGTGGACTGCGCCCTCGACCCAC
TGAGCGAGACAAAGTGCACACTGAAGTCTTTCACAGTGGAGAAGGGCATCTACCA
GACATCTAACTTCAGAGTGCAGCCTACAGAGTCTATCGTGAGATTCCCAAACATCA
CAAACCTGTGCCCTTTCGGCGAGGTGTTCAACGCTACACGGTTCGCTTCTGTGTAC
GCTTGGAACCGGAAGCGGATCTCTAACTGCGTGGCCGACTACTCTGTGCTGTACAA
CTCCGCCTCTTTCTCCACATTCAAGTGCTACGGCGTGAGCCCAACAAAGCTGAACG
ACCTGTGCTTCACAAACGTGTACGCCGACTCTTTCGTGATCCGGGGCGACGAGGTG
AGGCAGATCGCTCCAGGCCAGACAGGCAAGATCGCCGACTACAACTACAAGCTCC
CCGACGACTTCACAGGCTGCGTGATCGCTTGGAACTCTAACAACCTGGACTCTAAG
GTGGGCGGCAACTACAACTACCTGTACAGACTGTTCCGGAAGTCTAACCTGAAGC
CTTTCGAGCGGGACATCTCTACCGAGATCTACCAGGCCGGCAGCACCCCTTGCAAC
GGCGTGGAGGGCTTCAACTGCTACTTCCCACTGCAGTCTTACGGCTTCCAGCCTAC
AAACGGCGTGGGCTACCAGCCATACCGGGTGGTGGTGCTGTCCTTCGAGCTCCTCC
ACGCCCCCGCTACAGTGTGCGGCCCAAAGAAGTCCACAAACCTCGTGAAGAACAA
GTGCGTGAACTTCAACTTCAACGGCCTGACAGGCACAGGCGTGCTCACAGAGTCT
AACAAGAAGTTCCTCCCTTTCCAGCAGTTCGGCCGGGACATCGCCGACACCACCG
ACGCCGTGAGGGACCCTCAGACACTCGAGATCCTCGACATCACCCCTTGCTCTTTC
GGCGGCGTGTCTGTGATCACCCCCGGCACAAACACATCTAACCAGGTGGCTGTGCT
GTACCAGGACGTGAACTGCACCGAGGTGCCAGTGGCTATCCACGCCGACCAGCTC
ACCCCTACATGGAGGGTGTACAGCACAGGCTCTAACGTGTTCCAGACGAGGGCCG
GCTGCCTCATCGGCGCCGAGCACGTGAACAACTCTTACGAGTGCGACATCCCAATC
GGCGCTGGCATCTGCGCTTCTTACCAGACCCAGACAAACAGCCCTAGGAGAGCTA
GGTCCGTGGCTAGCCAGTCCATCATCGCTTACACCATGTCTCTGGGCGCTGAGAAC
TCTGTGGCTTACTCTAACAACTCTATCGCTATCCCTACAAACTTCACAATCTCTGTG
ACCACCGAGATCCTGCCAGTGTCTATGACAAAGACATCCGTGGACTGCACCATGT
ACATCTGCGGCGACAGCACCGAGTGCAGCAACCTGCTCCTGCAGTACGGCTCTTTC
TGCACACAGCTGAACCGGGCCCTCACCGGCATCGCCGTGGAGCAGGACAAGAACA
CCCAGGAGGTGTTCGCTCAGGTGAAGCAGATCTACAAGACCCCCCCTATCAAGGA
CTTCGGCGGCTTCAACTTCTCTCAGATCCTCCCCGACCCTAGCAAGCCTAGCAAGC
GGTCTTTCATCGAGGACCTCCTGTTCAACAAGGTGACACTCGCTGACGCTGGCTTC
ATCAAGCAGTACGGCGACTGCCTGGGCGACATCGCCGCTAGAGACCTGATCTGCG
CTCAGAAGTTCAACGGCCTCACAGTGCTGCCCCCACTCCTGACCGACGAGATGATC
GCTCAGTACACCAGCGCCCTCCTCGCTGGCACAATCACATCTGGCTGGACATTCGG
CGCCGGCGCCGCCCTGCAGATCCCATTCGCCATGCAGATGGCTTACCGGTTCAACG
GCATCGGCGTGACCCAGAACGTGCTGTACGAGAACCAGAAGCTGATCGCTAACCA
GTTCAACTCCGCTATCGGCAAGATCCAGGACTCCCTGTCTAGCACCGCTAGCGCCC
TGGGCAAGCTCCAGGACGTGGTGAACCAGAACGCCCAGGCCCTGAACACACTGGT
GAAGCAGCTGTCTTCTAACTTCGGCGCTATCAGCTCTGTGCTCAACGACATCCTGT
CTAGACTCGACAAGGTGGAGGCCGAGGTGCAGATCGACAGACTGATCACAGGCAG
ACTGCAGTCCCTCCAGACATACGTGACCCAGCAGCTCATCCGGGCCGCTGAGATC
AGAGCTAGCGCTAACCTCGCCGCTACCAAGATGAGCGAGTGCGTGCTGGGCCAGT
CTAAGCGGGTGGACTTCTGCGGCAAGGGCTACCACCTCATGTCTTTCCCTCAGTCC
GCCCCTCACGGCGTGGTGTTCCTCCACGTGACATACGTGCCAGCTCAGGAGAAGA
ACTTCACCACCGCCCCAGCTATCTGCCACGACGGCAAGGCCCACTTCCCACGGGA
GGGCGTGTTCGTGTCTAACGGCACCCACTGGTTCGTGACACAGCGGAACTTCTACG
AGCCTCAGATCATCACCACAGACAACACATTCGTGTCCGGCAACTGCGACGTGGT
GATCGGCATCGTGAACAACACAGTGTACGACCCACTGCAGCCCGAGCTCGACTCT
TTCAAGGAGGAGCTCGACAAGTACTTCAAGAACCACACCAGCCCCGACGTGGACC
TGGGCGACATCTCTGGCATCAACGCCTCTGTGGTGAACATCCAGAAGGAGATCGA
CAGACTGAACGAGGTGGCTAAGAACCTGAACGAGTCCCTGATCGACCTGCAGGAG
CTGGGCAAGTACGAGCAGTACATCAAGTGGCCTTGGTACATCTGGCTGGGCTTCAT
CGCTGGCCTGATCGCTATCGTGATGGTGACAATCATGCTGTGCTGCATGACCTCTT
GCTGCTCTTGCCTGAAGGGCTGCTGCTCTTGCGGCTCTTGCTGCAAGTTCGACGAG
GACGACTCCGAGCCCGTGCTGAAGGGCGTGAAGCTCCACTACACATGA
truncated S protein coding sequence
(S-dTM-opt, SEQ ID NO: 4)
ATGTTCGTGTTCCTGGTGCTCCTCCCTCTCGTGTCTTCTCAGTGCGTGAACCTG
ACCACACGGACCCAGCTGCCACCCGCTTACACCAACTCCTTCACAAGAGGCGTGT
ACTACCCCGACAAGGTGTTCCGGTCTTCTGTGCTCCACTCTACCCAGGACCTGTTC
CTGCCATTCTTCTCTAACGTGACATGGTTCCACGCTATCCACGTGTCTGGCACCAA
CGGCACAAAGAGATTCGACAACCCCGTGCTCCCATTCAACGACGGCGTGTACTTC
GCTAGCACAGAGAAGTCCAACATCATCCGGGGCTGGATCTTCGGCACCACACTGG
ACTCTAAGACCCAGTCCCTCCTCATCGTGAACAACGCCACAAACGTGGTGATCAA
GGTGTGCGAGTTCCAGTTCTGCAACGACCCATTCCTGGGCGTGTACTACCACAAGA
ACAACAAGTCTTGGATGGAGAGCGAGTTCCGGGTGTACTCTAGCGCTAACAACTG
CACATTCGAGTACGTGTCTCAGCCATTCCTCATGGACCTCGAGGGCAAGCAGGGC
AACTTCAAGAACCTGAGGGAGTTCGTGTTCAAGAACATCGACGGCTACTTCAAGA
TCTACTCTAAGCACACCCCAATCAACCTGGTGAGGGACCTGCCTCAGGGCTTCTCC
GCTCTCGAGCCACTGGTGGACCTGCCAATCGGCATCAACATCACCCGGTTCCAGAC
ACTCCTCGCCCTCCACCGGTCTTACCTGACCCCAGGCGACTCTAGCAGCGGCTGGA
CCGCCGGCGCCGCCGCTTACTACGTGGGCTACCTGCAGCCTAGGACATTCCTCCTG
AAGTACAACGAGAACGGCACAATCACCGACGCTGTGGACTGCGCCCTCGACCCAC
TGAGCGAGACAAAGTGCACACTGAAGTCTTTCACAGTGGAGAAGGGCATCTACCA
GACATCTAACTTCAGAGTGCAGCCTACAGAGTCTATCGTGAGATTCCCAAACATCA
CAAACCTGTGCCCTTTCGGCGAGGTGTTCAACGCTACACGGTTCGCTTCTGTGTAC
GCTTGGAACCGGAAGCGGATCTCTAACTGCGTGGCCGACTACTCTGTGCTGTACAA
CTCCGCCTCTTTCTCCACATTCAAGTGCTACGGCGTGAGCCCAACAAAGCTGAACG
ACCTGTGCTTCACAAACGTGTACGCCGACTCTTTCGTGATCCGGGGCGACGAGGTG
AGGCAGATCGCTCCAGGCCAGACAGGCAAGATCGCCGACTACAACTACAAGCTCC
CCGACGACTTCACAGGCTGCGTGATCGCTTGGAACTCTAACAACCTGGACTCTAAG
GTGGGCGGCAACTACAACTACCTGTACAGACTGTTCCGGAAGTCTAACCTGAAGC
CTTTCGAGCGGGACATCTCTACCGAGATCTACCAGGCCGGCAGCACCCCTTGCAAC
GGCGTGGAGGGCTTCAACTGCTACTTCCCACTGCAGTCTTACGGCTTCCAGCCTAC
AAACGGCGTGGGCTACCAGCCATACCGGGTGGTGGTGCTGTCCTTCGAGCTCCTCC
ACGCCCCCGCTACAGTGTGCGGCCCAAAGAAGTCCACAAACCTCGTGAAGAACAA
GTGCGTGAACTTCAACTTCAACGGCCTGACAGGCACAGGCGTGCTCACAGAGTCT
AACAAGAAGTTCCTCCCTTTCCAGCAGTTCGGCCGGGACATCGCCGACACCACCG
ACGCCGTGAGGGACCCTCAGACACTCGAGATCCTCGACATCACCCCTTGCTCTTTC
GGCGGCGTGTCTGTGATCACCCCCGGCACAAACACATCTAACCAGGTGGCTGTGCT
GTACCAGGACGTGAACTGCACCGAGGTGCCAGTGGCTATCCACGCCGACCAGCTC
ACCCCTACATGGAGGGTGTACAGCACAGGCTCTAACGTGTTCCAGACGAGGGCCG
GCTGCCTCATCGGCGCCGAGCACGTGAACAACTCTTACGAGTGCGACATCCCAATC
GGCGCTGGCATCTGCGCTTCTTACCAGACCCAGACAAACAGCCCTAGGAGAGCTA
GGTCCGTGGCTAGCCAGTCCATCATCGCTTACACCATGTCTCTGGGCGCTGAGAAC
TCTGTGGCTTACTCTAACAACTCTATCGCTATCCCTACAAACTTCACAATCTCTGTG
ACCACCGAGATCCTGCCAGTGTCTATGACAAAGACATCCGTGGACTGCACCATGT
ACATCTGCGGCGACAGCACCGAGTGCAGCAACCTGCTCCTGCAGTACGGCTCTTTC
TGCACACAGCTGAACCGGGCCCTCACCGGCATCGCCGTGGAGCAGGACAAGAACA
CCCAGGAGGTGTTCGCTCAGGTGAAGCAGATCTACAAGACCCCCCCTATCAAGGA
CTTCGGCGGCTTCAACTTCTCTCAGATCCTCCCCGACCCTAGCAAGCCTAGCAAGC
GGTCTTTCATCGAGGACCTCCTGTTCAACAAGGTGACACTCGCTGACGCTGGCTTC
ATCAAGCAGTACGGCGACTGCCTGGGCGACATCGCCGCTAGAGACCTGATCTGCG
CTCAGAAGTTCAACGGCCTCACAGTGCTGCCCCCACTCCTGACCGACGAGATGATC
GCTCAGTACACCAGCGCCCTCCTCGCTGGCACAATCACATCTGGCTGGACATTCGG
CGCCGGCGCCGCCCTGCAGATCCCATTCGCCATGCAGATGGCTTACCGGTTCAACG
GCATCGGCGTGACCCAGAACGTGCTGTACGAGAACCAGAAGCTGATCGCTAACCA
GTTCAACTCCGCTATCGGCAAGATCCAGGACTCCCTGTCTAGCACCGCTAGCGCCC
TGGGCAAGCTCCAGGACGTGGTGAACCAGAACGCCCAGGCCCTGAACACACTGGT
GAAGCAGCTGTCTTCTAACTTCGGCGCTATCAGCTCTGTGCTCAACGACATCCTGT
CTAGACTCGACAAGGTGGAGGCCGAGGTGCAGATCGACAGACTGATCACAGGCAG
ACTGCAGTCCCTCCAGACATACGTGACCCAGCAGCTCATCCGGGCCGCTGAGATC
AGAGCTAGCGCTAACCTCGCCGCTACCAAGATGAGCGAGTGCGTGCTGGGCCAGT
CTAAGCGGGTGGACTTCTGCGGCAAGGGCTACCACCTCATGTCTTTCCCTCAGTCC
GCCCCTCACGGCGTGGTGTTCCTCCACGTGACATACGTGCCAGCTCAGGAGAAGA
ACTTCACCACCGCCCCAGCTATCTGCCACGACGGCAAGGCCCACTTCCCACGGGA
GGGCGTGTTCGTGTCTAACGGCACCCACTGGTTCGTGACACAGCGGAACTTCTACG
AGCCTCAGATCATCACCACAGACAACACATTCGTGTCCGGCAACTGCGACGTGGT
GATCGGCATCGTGAACAACACAGTGTACGACCCACTGCAGCCCGAGCTCGACTCT
TTCAAGGAGGAGCTCGACAAGTACTTCAAGAACCACACCAGCCCCGACGTGGACC
TGGGCGACATCTCTGGCATCAACGCCTCTGTGGTGAACATCCAGAAGGAGATCGA
CAGACTGAACGAGGTGGCTAAGAACCTGAACGAGTCCCTGATCGACCTGCAGGAG
CTGGGCAAGTACGAGCAGTACATCAAGTGGCCTTGA
amino acid sequence of S1 subunit
(SEQ ID NO: 7)
MFVFLVLLPLVSSQCVNLTTRTQLPPAYTNSFTRGVYYPDKVFRSSVLHSTQDLFL
PFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTITDAV
DCALDPLSETKCTLKSFTVEKGIYQTSNFRVQPTESIVRFPNITNLCPFGEVENATRFAS
VYAWNRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKSNLKPF
ERDISTEIYQAGSTPCNGVEGENCYFPLQSYGFQPTNGVGYQPYRVVVLSFELLHAPAT
VCGPKKSTNLVKNKCVNFNFNGLTGTGVLTESNKKFLPFQQFGRDIADTTDAVRDPQ
TLEILDITPCSFGGVSVITPGTNTSNQVAVLYQDVNCTEVPVAIHADQLTPTWRVYSTG
SNVFQTRAGCLIGAEHVNNSYECDIPIGAGICASYQTQTNSPRRAR.
pCW1093 full length sequence
(8515 bp, SEQ ID NO: 11)
CTGACGTCGCGGTCGACAATATTGGCTATTGGCCATTGCATACGTTGTATCTA
TATCATAATATGTACATTTATATTGGCTCATGTCCAATATGACCGCCATGTTGACAT
TAGTTATTGACTAGTTATTAATAGTAATCAATTACGGGGTCATTAGTTCATAGCCC
ATATATGGAGTTCCGCGTTACATAACTTACGGTAAATGGCCCGCCTGGCTGACCGC
CCAACGACCCCCGCCCATTGACGTCAATAATGACGTATGTTCCCATAGTAACGCCA
ATAGGGACTTTCCATTGACGTCAATGGGTGGAGTATTTACGGTAAACTGCCCACTT
GGCAGTACATCAAGTGTATCATATGCCAAGTCCGCCCCCTATTGACGTCAATGACG
GTAAATGGCCCGCCTGGCATTATGCCCAGTACATGACCTTACGGGACTTTCCTACT
TGGCAGTACATCTACGTATTAGTCATCGCTATTACCATGGTGATGCGGTTTTGGCA
GTACACCAATGGGCGTGGATAGCGGTTTGACTCACGGGGATTTCCAAGTCTCCACC
CCATTGACGTCAATGGGAGTTTGTTTTGGCACCAAAATCAACGGGACTTTCCAAAA
TGTCGTAATAACCCCGCCCCGTTGACGCAAATGGGCGGTAGGCGTGTACGGTGGG
AGGTCTATATAAGCAGAGCTCGTTTAGTGAACCGTCAGATCGCCTGGAGACGCCA
TCCACGCTGTTTTGACCTCCATAGAAGACACCGGGACCGATCCAGCCTCCGCGGCC
GGGAACGGTGCATTGGAACGCGGATTCCCCGTGCCAAGAGTGACGTAAGTACCGC
CTATAGACTCTATAGGCACACCCCTTTGGCTCTTATGCATGCTATACTGTTTTTGGC
TTGGGGCCTATACACCCCCGCTTCCTTATGCTATAGGTGATGGTATAGCTTAGCCT
ATAGGTGTGGGTTATTGACCATTATTGACCACTCCCCTATTGGTGACGATACTTTCC
ATTACTAATCCATAACATGGCTCTTTGCCACAACTATCTCTATTGGCTATATGCCAA
TACTCTGTCCTTCAGAGACTGACACGGACTCTGTATTTTTACAGGATGGGGTCCCA
TTTATTATTTACAAATTCACATATACAACAACGCCGTCCCCCGTGCCCGCAGTTTTT
ATTAAACATAGCGTGGGATCTCCACGCGAATCTCGGGTACGTGTTCCGGACATGG
GCTCTTCTCCGGTAGCGGCGGAGCTTCCACATCCGAGCCCTGGTCCCATGCCTCCA
GCGGCTCATGGTCGCTCGGCAGCTCCTTGCTCCTAACAGTGGAGGCCAGACTTAGG
CACAGCACAATGCCCACCACCACCAGTGTGCCGCACAAGGCCGTGGCGGTAGGGT
ATGTGTCTGAAAATGAGCTCGGAGATTGGGCTCGCACCGCTGACGCAGATGGAAG
ACTTAAGGCAGCGGCAGAAGAAGATGCAGGCAGCTGAGTTGTTGTATTCTGATAA
GAGTCAGAGGTAACTCCCGTTGCGGTGCTGTTAACGGTGGAGGGCAGTGTAGTCT
GAGCAGTACTCGTTGCTGCCGCGCGCGCCACCAGACATAATAGCTGACAGACTAA
CAGACTGTTCCTTTCCATGGGTCTTTTCTGCAGTCACCGTCCAAGCTTGCAATCGCC
ACCATGTTCGTGTTCCTGGTGCTCCTCCCTCTCGTGTCTTCTCAGTGCGTGAACCTG
ACCACACGGACCCAGCTGCCACCCGCTTACACCAACTCCTTCACAAGAGGCGTGT
ACTACCCCGACAAGGTGTTCCGGTCTTCTGTGCTCCACTCTACCCAGGACCTGTTC
CTGCCATTCTTCTCTAACGTGACATGGTTCCACGCTATCCACGTGTCTGGCACCAA
CGGCACAAAGAGATTCGACAACCCCGTGCTCCCATTCAACGACGGCGTGTACTTC
GCTAGCACAGAGAAGTCCAACATCATCCGGGGCTGGATCTTCGGCACCACACTGG
ACTCTAAGACCCAGTCCCTCCTCATCGTGAACAACGCCACAAACGTGGTGATCAA
GGTGTGCGAGTTCCAGTTCTGCAACGACCCATTCCTGGGCGTGTACTACCACAAGA
ACAACAAGTCTTGGATGGAGAGCGAGTTCCGGGTGTACTCTAGCGCTAACAACTG
CACATTCGAGTACGTGTCTCAGCCATTCCTCATGGACCTCGAGGGCAAGCAGGGC
AACTTCAAGAACCTGAGGGAGTTCGTGTTCAAGAACATCGACGGCTACTTCAAGA
TCTACTCTAAGCACACCCCAATCAACCTGGTGAGGGACCTGCCTCAGGGCTTCTCC
GCTCTCGAGCCACTGGTGGACCTGCCAATCGGCATCAACATCACCCGGTTCCAGAC
ACTCCTCGCCCTCCACCGGTCTTACCTGACCCCAGGCGACTCTAGCAGCGGCTGGA
CCGCCGGCGCCGCCGCTTACTACGTGGGCTACCTGCAGCCTAGGACATTCCTCCTG
AAGTACAACGAGAACGGCACAATCACCGACGCTGTGGACTGCGCCCTCGACCCAC
TGAGCGAGACAAAGTGCACACTGAAGTCTTTCACAGTGGAGAAGGGCATCTACCA
GACATCTAACTTCAGAGTGCAGCCTACAGAGTCTATCGTGAGATTCCCAAACATCA
CAAACCTGTGCCCTTTCGGCGAGGTGTTCAACGCTACACGGTTCGCTTCTGTGTAC
GCTTGGAACCGGAAGCGGATCTCTAACTGCGTGGCCGACTACTCTGTGCTGTACAA
CTCCGCCTCTTTCTCCACATTCAAGTGCTACGGCGTGAGCCCAACAAAGCTGAACG
ACCTGTGCTTCACAAACGTGTACGCCGACTCTTTCGTGATCCGGGGCGACGAGGTG
AGGCAGATCGCTCCAGGCCAGACAGGCAAGATCGCCGACTACAACTACAAGCTCC
CCGACGACTTCACAGGCTGCGTGATCGCTTGGAACTCTAACAACCTGGACTCTAAG
GTGGGCGGCAACTACAACTACCTGTACAGACTGTTCCGGAAGTCTAACCTGAAGC
CTTTCGAGCGGGACATCTCTACCGAGATCTACCAGGCCGGCAGCACCCCTTGCAAC
GGCGTGGAGGGCTTCAACTGCTACTTCCCACTGCAGTCTTACGGCTTCCAGCCTAC
AAACGGCGTGGGCTACCAGCCATACCGGGTGGTGGTGCTGTCCTTCGAGCTCCTCC
ACGCCCCCGCTACAGTGTGCGGCCCAAAGAAGTCCACAAACCTCGTGAAGAACAA
GTGCGTGAACTTCAACTTCAACGGCCTGACAGGCACAGGCGTGCTCACAGAGTCT
AACAAGAAGTTCCTCCCTTTCCAGCAGTTCGGCCGGGACATCGCCGACACCACCG
ACGCCGTGAGGGACCCTCAGACACTCGAGATCCTCGACATCACCCCTTGCTCTTTC
GGCGGCGTGTCTGTGATCACCCCCGGCACAAACACATCTAACCAGGTGGCTGTGCT
GTACCAGGACGTGAACTGCACCGAGGTGCCAGTGGCTATCCACGCCGACCAGCTC
ACCCCTACATGGAGGGTGTACAGCACAGGCTCTAACGTGTTCCAGACGAGGGCCG
GCTGCCTCATCGGCGCCGAGCACGTGAACAACTCTTACGAGTGCGACATCCCAATC
GGCGCTGGCATCTGCGCTTCTTACCAGACCCAGACAAACAGCCCTAGGAGAGCTA
GGTCCGTGGCTAGCCAGTCCATCATCGCTTACACCATGTCTCTGGGCGCTGAGAAC
TCTGTGGCTTACTCTAACAACTCTATCGCTATCCCTACAAACTTCACAATCTCTGTG
ACCACCGAGATCCTGCCAGTGTCTATGACAAAGACATCCGTGGACTGCACCATGT
ACATCTGCGGCGACAGCACCGAGTGCAGCAACCTGCTCCTGCAGTACGGCTCTTTC
TGCACACAGCTGAACCGGGCCCTCACCGGCATCGCCGTGGAGCAGGACAAGAACA
CCCAGGAGGTGTTCGCTCAGGTGAAGCAGATCTACAAGACCCCCCCTATCAAGGA
CTTCGGCGGCTTCAACTTCTCTCAGATCCTCCCCGACCCTAGCAAGCCTAGCAAGC
GGTCTTTCATCGAGGACCTCCTGTTCAACAAGGTGACACTCGCTGACGCTGGCTTC
ATCAAGCAGTACGGCGACTGCCTGGGCGACATCGCCGCTAGAGACCTGATCTGCG
CTCAGAAGTTCAACGGCCTCACAGTGCTGCCCCCACTCCTGACCGACGAGATGATC
GCTCAGTACACCAGCGCCCTCCTCGCTGGCACAATCACATCTGGCTGGACATTCGG
CGCCGGCGCCGCCCTGCAGATCCCATTCGCCATGCAGATGGCTTACCGGTTCAACG
GCATCGGCGTGACCCAGAACGTGCTGTACGAGAACCAGAAGCTGATCGCTAACCA
GTTCAACTCCGCTATCGGCAAGATCCAGGACTCCCTGTCTAGCACCGCTAGCGCCC
TGGGCAAGCTCCAGGACGTGGTGAACCAGAACGCCCAGGCCCTGAACACACTGGT
GAAGCAGCTGTCTTCTAACTTCGGCGCTATCAGCTCTGTGCTCAACGACATCCTGT
CTAGACTCGACAAGGTGGAGGCCGAGGTGCAGATCGACAGACTGATCACAGGCAG
ACTGCAGTCCCTCCAGACATACGTGACCCAGCAGCTCATCCGGGCCGCTGAGATC
AGAGCTAGCGCTAACCTCGCCGCTACCAAGATGAGCGAGTGCGTGCTGGGCCAGT
CTAAGCGGGTGGACTTCTGCGGCAAGGGCTACCACCTCATGTCTTTCCCTCAGTCC
GCCCCTCACGGCGTGGTGTTCCTCCACGTGACATACGTGCCAGCTCAGGAGAAGA
ACTTCACCACCGCCCCAGCTATCTGCCACGACGGCAAGGCCCACTTCCCACGGGA
GGGCGTGTTCGTGTCTAACGGCACCCACTGGTTCGTGACACAGCGGAACTTCTACG
AGCCTCAGATCATCACCACAGACAACACATTCGTGTCCGGCAACTGCGACGTGGT
GATCGGCATCGTGAACAACACAGTGTACGACCCACTGCAGCCCGAGCTCGACTCT
TTCAAGGAGGAGCTCGACAAGTACTTCAAGAACCACACCAGCCCCGACGTGGACC
TGGGCGACATCTCTGGCATCAACGCCTCTGTGGTGAACATCCAGAAGGAGATCGA
CAGACTGAACGAGGTGGCTAAGAACCTGAACGAGTCCCTGATCGACCTGCAGGAG
CTGGGCAAGTACGAGCAGTACATCAAGTGGCCTTGGTACATCTGGCTGGGCTTCAT
CGCTGGCCTGATCGCTATCGTGATGGTGACAATCATGCTGTGCTGCATGACCTCTT
GCTGCTCTTGCCTGAAGGGCTGCTGCTCTTGCGGCTCTTGCTGCAAGTTCGACGAG
GACGACTCCGAGCCCGTGCTGAAGGGCGTGAAGCTCCACTACACATGATAAGGAT
CCTCGCAATCCCTAGGAGGATTAGGCAAGGGCTTGAGCTCACGCTCTTGTGAGGG
ACAGAAATACAATCAGGGGCAGTATATGAATACTCCATGGAGAAACCCAGATCTA
CGTATGATCAGCCTCGACTGTGCCTTCTAGTTGCCAGCCATCTGTTGTTTGCCCCTC
CCCCGTGCCTTCCTTGACCCTGGAAGGTGCCACTCCCACTGTCCTTTCCTAATAAA
ATGAGGAAATTGCATCGCATTGTCTGAGTAGGTGTCATTCTATTCTGGGGGGTGGG
GTGGGGCAGGACAGCAAGGGGGAGGATTGGGAAGACAATAGCAGGCATGCTGGG
GATGCGGTGGGCTCTATGGCTTCTGAGGCGGAAAGAACCAGCTGGGGCTCGACAG
CTCGAGCTAGGACCGCTATCAGGACATAGCGTTGGCTACCCGTGATATTGCTGAAG
AGCTTGGCGGCGAATGGGCTGACCGCTTCCTCGTGCTTTACGGTATCGCCGCTCCC
GATTCGCAGCGCATCGCCTTCTATCGCCTTCTTGACGAGTTCTTCTGAGCGGGACT
CTGGGGTTCGAAATGACCGACCAAGCGACGCCCAACCTGCCATCACGAGATTTCG
ATTCCACCGCCGCCTTCTATGAAAGGTTGGGCTTCGGAATCGTTTTCCGGGACGCC
GGCTGGATGATCCTCCAGCGCGGGGATCTCATGCTGGAGTTCTTCGCCCACCCCAA
CTTGTTTATTGCAGCTTATAATGGTTACAAATAAAGCAATAGCATCACAAATTTCA
CAAATAAAGCATTTTTTTCACTGCATTCTAGTTGTGGTTTGTCCAAACTCATCAATG
TATCTTATCATGTCTGGATCGCGGCCGCGATCCGTCGAGAGCTTGGCGTAATCATG
GTCATAGCTGTTTCCTGTGTGAAATTGTTATCCGCTCACAATTCCACACAACATAC
GAGCCGGAAGCATAAAGTGTAAAGCCTGGGGTGCCTAATGAGTGAGCTAACTCAC
ATTAATTGCGTTGCGCTCACTGCCCGCTTTCCAGTCGGGAAACCTGTCGTGCCAGC
TGCATTAATGAATCGGCCAACGCGCGGGGAGAGGCGGTTTGCGTATTGGGCGCTC
TTCCGCTTCCTCGCTCACTGACTCGCTGCGCTCGGTCGTTCGGCTGCGGCGAGCGG
TATCAGCTCACTCAAAGGCGGTAATACGGTTATCCACAGAATCAGGGGATAACGC
AGGAAAGAACATGTGAGCAAAAGGCCAGCAAAAGGCCAGGAACCGTAAAAAGGC
CGCGTTGCTGGCGTTTTTCCATAGGCTCCGCCCCCCTGACGAGCATCACAAAAATC
GACGCTCAAGTCAGAGGTGGCGAAACCCGACAGGACTATAAAGATACCAGGCGTT
TCCCCCTGGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCACGCTGTAG
GTATCTCAGTTCGGTGTAGGTCGTTCGCTCCAAGCTGGGCTGTGTGCACGAACCCC
CCGTTCAGCCCGACCGCTGCGCCTTATCCGGTAACTATCGTCTTGAGTCCAACCCG
GTAAGACACGACTTATCGCCACTGGCAGCAGCCACTGGTAACAGGATTAGCAGAG
CGAGGTATGTAGGCGGTGCTACAGAGTTCTTGAAGTGGTGGCCTAACTACGGCTA
CACTAGAAGAACAGTATTTGGTATCTGCGCTCTGCTGAAGCCAGTTACCTTCGGAA
AAAGAGTTGGTAGCTCTTGATCCGGCAAACAAACCACCGCTGGTAGCGGTGGTTTT
TTTGTTTGCAAGCAGCAGATTACGCGCAGAAAAAAAGGATCTCAAGAAGATCCTT
TGATCTTTTCTACGGGGTCTGACGCTCAGTGGAACGAAAACTCACGTTAAGGGATT
TTGGTCATGAGATTATCAAAAAGGATCTTCACCTAGATCCTTTTAAATTAAAAATG
AAGTTTTAAATCAATCTAAAGTATATATGAGTAAACTTGGTCTGACAGTCTAGCTT
AGAAAAACTCATCGAGCATCAAATGAAACTGCAATTTATTCATATCAGGATTATCA
ATACCATATTTTTGAAAAAGCCGTTTCTGTAATGAAGGAGAAAACTCACCGAGGC
AGTTCCATAGGATGGCAAGATCCTGGTATCGGTCTGCGATTCCGACTCGTCCAACA
TCAATACAACCTATTAATTTCCCCTCGTCAAAAATAAGGTTATCAAGTGAGAAATC
ACCATGAGTGACGACTGAATCCGGTGAGAATGGCAAAAGCTTATGCATTTCTTTCC
AGACTTGTTCAACAGGCCAGCCATTACGCTCGTCATCAAAATCACTCGCATCAACC
AAACCGTTATTCATTCGTGATTGCGCCTGAGCGAGACGAAATACGCGATCGCTGTT
AAAAGGACAATTACAAACAGGAATCGAATGCAACCGGCGCAGGAACACTGCCAG
CGCATCAACAATATTTTCACCTGAATCAGGATATTCTTCTAATACCTGGAATGCTG
TTTTCCCGGGGATCGCAGTGGTGAGTAACCATGCATCATCAGGAGTACGGATAAA
ATGCTTGATGGTCGGAAGAGGCATAAATTCCGTCAGCCAGTTTAGTCTGACCATCT
CATCTGTAACATCATTGGCAACGCTACCTTTGCCATGTTTCAGAAACAACTCTGGC
GCATCGGGCTTCCCATACAATCGATAGATTGTCGCACCTGATTGCCCGACATTATC
GCGAGCCCATTTATACCCATATAAATCAGCATCCATGTTGGAATTTAATCGCGGCC
TCGAGCAAGACGTTTCCCGTTGAATATGGCTCATGCTAGAACTCTTCCTTTTTCAAT
ATTATTGAAGCATTTATCAGGGTTATTGTCTCATGAGCGGATACATATTTGAATGT
ATTTAGAAAAATAAACAAATAGGGGTTCCGCGCACATTTCCCCGAAAAGTGCCAC

### REFERENCES

1. WHO. 2020. https://www.who.int/emergencies/diseases/novel-coronavirus-2019.
2. Abrams E M, Szefler S J. 2020. COVID-19 and the impact of social determinants of health. Lancet Respir Med 8: 659-61
3. Wiersinga W J, Rhodes A, Cheng A C, Peacock S J, Prescott H C. 2020. Pathophysiology, Transmission, Diagnosis, and Treatment of Coronavirus Disease 2019 (COVID-19): A Review. JAMA 324: 782-93
4. Chu D K, Akl E A, Duda S, Solo K, Yaacoub S, Schunemann H J, authors C-SURGEs. 2020. Physical distancing, face masks, and eye protection to prevent person-to-person transmission of SARS-CoV-2 and COVID-19: a systematic review and meta-analysis. Lancet 395: 1973-87
5. Bong C L, Brasher C, Chikumba E, McDougall R, Mellin-Olsen J, Enright A. 2020. The COVID-19 Pandemic: Effects on Low- and Middle-Income Countries. Anesth Analg 131: 86-92
6. Zar H J, Dawa J, Fischer G B, Castro-Rodriguez J A. 2020. Challenges of COVID-19 in children in low- and middle-income countries. Paediatr Respir Rev 35: 70-4
7. Chauhan S. 2020. Comprehensive review of coronavirus disease 2019 (COVID-19). Biomed J
8. Yang J, Niu P, Chen L, Wang L, Zhao L, Huang B, Ma J, Hu S, Wu L, Wu G, Huang C, Bi Y, Tan W. 2020. Genetic tracing of HCoV-19 for the re-emerging outbreak of COVID-19 in Beijing, China. Protein Cell
9. Kaur S P, Gupta V. 2020. COVID-19 Vaccine: A comprehensive status report. Virus Res 288: 198114
10. Lu S. 2020. Timely development of vaccines against SARS-CoV-2. Emerg Microbes Infect 9: 542-4
11. Bloom B R, Nowak G J, Orenstein W. 2020. “When Will We Have a Vaccine?”—Understanding Questions and Answers about Covid-19 Vaccination. N Engl J Med
12. Heaton P M. 2020. The Covid-19 Vaccine-Development Multiverse. N Engl J Med
13. WHO. 2020. https://www.who.int/publications/m/item/draft-landscape-of-covid-19-candidate-vaccines.
14. Zhu F C, Guan X H, Li Y H, Huang J Y, Jiang T, Hou L H, Li J X, Yang B F, Wang L, Wang W J, Wu S P, Wang Z, Wu X H, Xu J J, Zhang Z, Jia S Y, Wang B S, Hu Y, Liu J J, Zhang J, Qian X A, Li Q, Pan H X, Jiang H D, Deng P, Gou J B, Wang X W, Wang X H, Chen W. 2020. Immunogenicity and safety of a recombinant adenovirus type-5-vectored COVID-19 vaccine in healthy adults aged 18 years or older: a randomised, double-blind, placebo-controlled, phase 2 trial. Lancet 396: 479-88
15. Zhu F C, Li Y H, Guan X H, Hou L H, Wang W J, Li J X, Wu S P, Wang B S, Wang Z, Wang L, Jia S Y, Jiang H D, Wang L, Jiang T, Hu Y, Gou J B, Xu S B, Xu J J, Wang X W, Wang W, Chen W. 2020. Safety, tolerability, and immunogenicity of a recombinant adenovirus type-5 vectored COVID-19 vaccine: a dose-escalation, open-label, non-randomised, first-in-human trial. Lancet 395: 1845-54
16. Graham S P, McLean R K, Spencer A J, Belij-Rammerstorfer S, Wright D, Ulaszewska M, Edwards J C, Hayes J W P, Martini V, Thakur N, Conceicao C, Dietrich I, Shelton H, Waters R, Ludi A, Wilsden G, Browning C, Bialy D, Bhat S, Stevenson-Leggett P, Hollinghurst P, Gilbride C, Pulido D, Moffat K, Sharpe H, Allen E, Mioulet V, Chiu C, Newman J, Asfor A S, Burman A, Crossley S, Huo J, Owens R J, Carroll M, Hammond J A, Tchilian E, Bailey D, Charleston B, Gilbert S C, Tuthill T J, Lambe T. 2020. Evaluation of the immunogenicity of prime-boost vaccination with the replication-deficient viral vectored COVID-19 vaccine candidate ChAdOxl nCoV-19. NPJ Vaccines 5: 69
17. van Doremalen N, Lambe T, Spencer A, Belij-Rammerstorfer S, Purushotham J N, Port J R, Avanzato V A, Bushmaker T, Flaxman A, Ulaszewska M, Feldmann F, Allen E R, Sharpe H, Schulz J, Holbrook M, Okumura A, Meade-White K, Perez-Perez L, Edwards N J, Wright D, Bissett C, Gilbride C, Williamson B N, Rosenke R, Long D, Ishwarbhai A, Kailath R, Rose L, Morris S, Powers C, Lovaglio J, Hanley P W, Scott D, Saturday G, de Wit E, Gilbert S C, Munster V J. 2020. ChAdOxl nCoV-19 vaccine prevents SARS-CoV-2 pneumonia in rhesus macaques. Nature
18. Folegatti P M, Ewer K J, Aley P K, Angus B, Becker S, Belij-Rammerstorfer S, Bellamy D, Bibi S, Bittaye M, Clutterbuck E A, Dold C, Faust S N, Finn A, Flaxman A L, Hallis B, Heath P, Jenkin D, Lazarus R, Makinson R, Minassian A M, Pollock K M, Ramasamy M, Robinson H, Snape M, Tarrant R, Voysey M, Green C, Douglas A D, Hill A V S, Lambe T, Gilbert S C, Pollard A J, Oxford CVTG. 2020. Safety and immunogenicity of the ChAdOxl nCoV-19 vaccine against SARS-CoV-2: a preliminary report of a phase 1/2, single-blind, randomised controlled trial. Lancet 396: 467-78
19. Corbett K S, Edwards D K, Leist S R, Abiona O M, Boyoglu-Barnum S, Gillespie R A, Himansu S, Schafer A, Ziwawo C T, DiPiazza A T, Dinnon K H, Elbashir S M, Shaw C A, Woods A, Fritch E J, Martinez D R, Bock K W, Minai M, Nagata B M, Hutchinson G B, Wu K, Henry C, Bahi K, Garcia-Dominguez D, Ma L, Renzi I, Kong W P, Schmidt S D, Wang L, Zhang Y, Phung E, Chang L A, Loomis R J, Altaras N E, Narayanan E, Metkar M, Presnyak V, Liu C, Louder M K, Shi W, Leung K, Yang E S, West A, Gully K L, Stevens L J, Wang N, Wrapp D, Doria-Rose N A, Stewart-Jones G, Bennett H, Alvarado G S, Nason M C, Ruckwardt T J, McLellan J S, Denison M R, Chappell J D, Moore I N, Morabito K M, Mascola J R, Baric R S, Carfi A, Graham B S. 2020. SARS-CoV-2 mRNA vaccine design enabled by prototype pathogen preparedness. Nature
20. Corbett K S, Edwards D, Leist S R, Abiona O M, Boyoglu-Barnum S, Gillespie R A, Himansu S, Schafer A, Ziwawo C T, DiPiazza A T, Dinnon K H, Elbashir S M, Shaw C A, Woods A, Fritch E J, Martinez D R, Bock K W, Minai M, Nagata B M, Hutchinson G B, Bahl K, Garcia-Dominguez D, Ma L, Renzi I, Kong W P, Schmidt S D, Wang L, Zhang Y, Stevens L J, Phung E, Chang L A, Loomis R J, Altaras N E, Narayanan E, Metkar M, Presnyak V, Liu C, Louder M K, Shi W, Leung K, Yang E S, West A, Gully K L, Wang N, Wrapp D, Doria-Rose N A, Stewart-Jones G, Bennett H, Nason M C, Ruckwardt T J, McLellan J S, Denison M R, Chappell J D, Moore I N, Morabito K M, Mascola J R, Baric R S, Carfi A, Graham B S. 2020. SARS-CoV-2 mRNA Vaccine Development Enabled by Prototype Pathogen Preparedness. bioRxiv
21. Corbett K S, Flynn B, Foulds K E, Francica J R, Boyoglu-Barnum S, Werner A P, Flach B, O'Connell S, Bock K W, Minai M, Nagata B M, Andersen H, Martinez D R, Noe A T, Douek N, Donaldson M M, Nji N N, Alvarado G S, Edwards D K, Flebbe D R, Lamb E, Doria-Rose N A, Lin B C, Louder M K, O'Dell S, Schmidt S D, Phung E, Chang L A, Yap C, Todd J M, Pessaint L, Van Ry A, Browne S, Greenhouse J, Putman-Taylor T, Strasbaugh A, Campbell T A, Cook A, Dodson A, Steingrebe K, Shi W, Zhang Y, Abiona O M, Wang L, Pegu A, Yang E S, Leung K, Zhou T, Teng I T, Widge A, Gordon I, Novik L, Gillespie R A, Loomis R J, Moliva J I, Stewart-Jones G, Himansu S, Kong W P, Nason M C, Morabito K M, Ruckwardt T J, Ledgerwood J E, Gaudinski M R, Kwong P D, Mascola J R, Carfi A, Lewis M G, Baric R S, McDermott A, Moore I N, Sullivan N J, Roederer M, Seder R A, Graham B S. 2020. Evaluation of the mRNA-1273 Vaccine against SARS-CoV-2 in Nonhuman Primates. N Engl J Med
22. Jackson L A, Roberts P C, Graham B S. 2020. A SARS-CoV-2 mRNA Vaccine—Preliminary Report. Reply. N Engl J Med 383: 1191-2
23. Mulligan M J, Lyke K E, Kitchin N, Absalon J, Gurtman A, Lockhart S, Neuzil K, Raabe V, Bailey R, Swanson K A, Li P, Koury K, Kalina W, Cooper D, Fontes-Garfias C, Shi P Y, Tureci O, Tompkins K R, Walsh E E, Frenck R, Falsey A R, Dormitzer P R, Gruber W C, Sahin U, Jansen K U. 2020. Phase I/I I study of COVID-19 RNA vaccine BNT162b1 in adults. Nature
24. Shin M D, Shukla S, Chung Y H, Beiss V, Chan S K, Ortega-Rivera O A, Wirth D M, Chen A, Sack M, Pokorski J K, Steinmetz N F. 2020. COVID-19 vaccine development and a potential nanomaterial path forward. Nat Nanotechnol 15: 646-55
25. Xia S, Duan K, Zhang Y, Zhao D, Zhang H, Xie Z, Li X, Peng C, Zhang Y, Zhang W, Yang Y, Chen W, Gao X, You W, Wang X, Wang Z, Shi Z, Wang Y, Yang X, Zhang L, Huang L, Wang Q, Lu J, Yang Y, Guo J, Zhou W, Wan X, Wu C, Wang W, Huang S, Du J, Meng Z, Pan A, Yuan Z, Shen S, Guo W, Yang X. 2020. Effect of an Inactivated Vaccine Against SARS-CoV-2 on Safety and Immunogenicity Outcomes: Interim Analysis of 2 Randomized Clinical Trials. JAMA
26. Mulligan M J. 2020. An Inactivated Virus Candidate Vaccine to Prevent COVID-19. JAMA
27. Gao Q, Bao L, Mao H, Wang L, Xu K, Yang M, Li Y, Zhu L, Wang N, Lv Z, Gao H, Ge X, Kan B, Hu Y, Liu J, Cai F, Jiang D, Yin Y, Qin C, Li J, Gong X, Lou X, Shi W, Wu D, Zhang H, Zhu L, Deng W, Li Y, Lu J, Li C, Wang X, Yin W, Zhang Y, Qin C. 2020. Development of an inactivated vaccine candidate for SARS-CoV-2. Science 369: 77-81
28. Shi Y, Wang N, Zou Q M. 2020. [Progress and challenge of vaccine development against 2019-novel coronavirus (2019-nCoV)]. Zhonghua Yu Fang Yi Xue Za Zhi 54: 614-9
29. Kapikian A Z, Mitchell R H, Chanock R M, Shvedoff R A, Stewart C E. 1969. An epidemiologic study of altered clinical reactivity to respiratory syncytial (R S) virus infection in children previously vaccinated with an inactivated R S virus vaccine. Am J Epidemiol 89: 405-21
30. Kim H W, Canchola J G, Brandt C D, Pyles G, Chanock R M, Jensen K, Parrott R H. 1969. Respiratory syncytial virus disease in infants despite prior administration of antigenic inactivated vaccine. Am J Epidemiol 89: 422-34
31. Tillett R L, Sevinsky J R, Hartley P D, Kerwin H, Crawford N, Gorzalski A, Laverdure C, Verma S C, Rossetto C C, Jackson D, Farrell M J, Van Hooser S, Pandori M. 2020. Genomic evidence for reinfection with SARS-CoV-2: a case study. Lancet Infect Dis
32. Muecksch F, Wise H, Batchelor B, Squires M, Semple E, Richardson C, McGuire J, Clearly S, Furrie E, Greig N, Hay G, Templeton K, Lorenzi J C C, Hatziioannou T, Jenks S, Bieniasz P D. 2020. Longitudinal analysis of serology and neutralizing antibody levels in COVID19 convalescents. J Infect Dis
33. Chan P K S, Lui G, Hachim A, Ko R L W, Boon S S, Li T, Kavian N, Luk F, Chen Z, Yau E M, Chan K H, Tsang C H, Cheng S M S, Chu D K W, Perera R, Ho W C S, Yeung A C M, Chow C, Poon L L M, Valkenburg S A, Hui D S C, Peiris M. 2020. Serologic Responses in Healthy Adult with SARS-CoV-2 Reinfection, Hong Kong, August 2020. Emerg Infect Dis 26
34. Wang S, Kennedy J S, West K, Montefiori D C, Coley S, Lawrence J, Shen S, Green S, Rothman A L, Ennis F A, Arthos J, Pal R, Markham P, Lu S. 2008. Cross-subtype antibody and cellular immune responses induced by a polyvalent DNA prime-protein boost HIV-1 vaccine in healthy human volunteers. Vaccine 26: 3947-57
35. Almansour I, Chen H, Wang S, Lu S. 2013. Cross reactivity of serum antibody responses elicited by DNA vaccines expressing H A antigens from H1N1 subtype influenza vaccines in the past 30 years. Hum Vaccin Immunother 9: 2049-59
36. Vaine M, Wang S, Hackett A, Arthos J, Lu S. 2010. Antibody responses elicited through homologous or heterologous prime-boost DNA and protein vaccinations differ in functional activity and avidity. Vaccine 28: 2999-3007
37. Wang S, Arthos J, Lawrence J M, Van Ryk D, Mboudjeka I, Shen S, Chou T H, Montefiori D C, Lu S. 2005. Enhanced immunogenicity of gp120 protein when combined with recombinant DNA priming to generate antibodies that neutralize the JR-FL primary isolate of human immunodeficiency virus type 1. J Virol 79: 7933-7
38. Tavel J A, Martin J E, Kelly G G, Enama M E, Shen J M, Gomez P L, Andrews C A, Koup R A, Bailer R T, Stein J A, Roederer M, Nabel G J, Graham B S. 2007. Safety and immunogenicity of a Gag-Pol candidate HIV-1 DNA vaccine administered by a needle-free device in HIV-1-seronegative subjects. J Acquir Immune Defic Syndr 44: 601-5
39. Jacobson J M, Zheng L, Wilson C C, Tebas P, Matining R M, Egan M A, Eldridge J, Landay A L, Clifford D B, Luetkemeyer A F, Tiu J, Martinez A L, Janik J, Spitz T A, Hural J, McElrath J, Frahm N, Team A A P. 2016. The Safety and Immunogenicity of an Interleukin-12-Enhanced Multiantigen DNA Vaccine Delivered by Electroporation for the Treatment of HIV-1 Infection. J Acquir Immune Defic Syndr 71: 163-71
40. Kalams S A, Parker S, Jin X, Elizaga M, Metch B, Wang M, Hural J, Lubeck M, Eldridge J, Cardinali M, Blattner W A, Sobieszczyk M, Suriyanon V, Kalichman A, Weiner D B, Baden L R, Network NHVT. 2012. Safety and immunogenicity of an HIV-1 gag DNA vaccine with or without IL-12 and/or IL-15 plasmid cytokine adjuvant in healthy, HIV-1 uninfected adults. PLoS One 7: e29231
41. Lu S. 2009. Heterologous prime-boost vaccination. Curr Opin Immunol 21: 346-51
42. Wang S, Parker C, Taaffe J, Solorzano A, Garcia-Sastre A, Lu S. 2008. Heterologous H A DNA vaccine prime—inactivated influenza vaccine boost is more effective than using DNA or inactivated vaccine alone in eliciting antibody responses against H1 or H3 serotype influenza viruses. Vaccine 26: 3626-33
43. Ledgerwood J E, Wei C J, Hu Z, Gordon I J, Enama M E, Hendel C S, McTamney P M, Pearce M B, Yassine H M, Boyington J C, Bailer R, Tumpey T M, Koup R A, Mascola J R, Nabel G J, Graham B S, Team VRCS. 2011. DNA priming and influenza vaccine immunogenicity: two phase 1 open label randomised clinical trials. Lancet Infect Dis 11: 916-24
44. Ledgerwood J E, Hu Z, Costner P, Yamshchikov G, Enama M E, Plummer S, Hendel C S, Holman L, Larkin B, Gordon I, Bailer R T, Poretz D M, Sarwar U, Kabadi A, Koup R, Mascola J R, Graham B S, Vrc, Teams VRCS. 2015. Phase I clinical evaluation of seasonal influenza hemagglutinin (H A) DNA vaccine prime followed by trivalent influenza inactivated vaccine (IIV3) boost. Contemp Clin Trials 44: 112-8
45. Wang S, Chou T H, Sakhatskyy P V, Huang S, Lawrence J M, Cao H, Huang X, Lu S. 2005. Identification of two neutralizing regions on the severe acute respiratory syndrome coronavirus spike glycoprotein produced from the mammalian expression system. J Virol 79: 1906-10
46. Wang S, Taaffe J, Parker C, Solorzano A, Cao H, Garcia-Sastre A, Lu S. 2006. Hemagglutinin (H A) proteins from H1 and H3 serotypes of influenza A viruses require different antigen designs for the induction of optimal protective antibody responses as studied by codon-optimized H A DNA vaccines. J Virol 80: 11628-37
47. Wang S, Farfan-Arribas D J, Shen S, Chou T H, Hirsch A, He F, Lu S. 2006. Relative contributions of codon usage, promoter efficiency and leader sequence to the antigen expression and immunogenicity of HIV-1 Env DNA vaccine. Vaccine 24: 4531-40
48. Song H C, Seo M Y, Stadler K, Yoo B J, Choo Q L, Coates S R, Uematsu Y, Harada T, Greer C E, Polo J M, Pileri P, Eickmann M, Rappuoli R, Abrignani S, Houghton M, Han J H. 2004. Synthesis and characterization of a native, oligomeric form of recombinant severe acute respiratory syndrome coronavirus spike glycoprotein. J Virol 78: 10328-35
49. Esposito D, Mehalko J, Drew M, Snead K, Wall V, Taylor T, Frank P, Denson J P, Hong M, Gulten G, Sadtler K, Messing S, Gillette W. 2020. Optimizing high-yield production of SARS-CoV-2 soluble spike trimers for serology assays. Protein Expr Purif 174: 105686
50. Hsieh C L, Goldsmith J A, Schaub J M, DiVenere A M, Kuo H C, Javanmardi K, Le K C, Wrapp D, Lee A G, Liu Y, Chou C W, Byrne P O, Hj orth C K, Johnson N V, Ludes-Meyers J, Nguyen A W, Park J, Wang N, Amengor D, Lavinder J J, Ippolito G C, Maynard J A, Finkelstein I J, McLellan J S. 2020. Structure-based design of prefusion-stabilized SARS-CoV-2 spikes. Science 369: 1501-5
51. Li J, Valentin A, Kulkarni V, Rosati M, Beach R K, Alicea C, Hannaman D, Reed S G, Felber B K, Pavlakis G N. 2013. HIV/SIV DNA vaccine combined with protein in a co-immunization protocol elicits highest humoral responses to envelope in mice and macaques. Vaccine 31: 3747-55
52. Liu S, Wang S, Lu S. 2016. DNA immunization as a technology platform for monoclonal antibody induction. Emerg Microbes Infect 5: e33
53. Liu S, Wang S, Lu S. 2018. Using DNA Immunization to Elicit Monoclonal Antibodies in Mice, Rabbits, and Humans. Hum Gene Ther 29: 997-1003
54. Munson P, Liu Y, Bratt D, Fuller J T, Hu X, Pavlakis G N, Felber B K, Mullins J I, Fuller D H. 2018. Therapeutic conserved elements (C E) DNA vaccine induces strong T-cell responses against highly conserved viral sequences during simian-human immunodeficiency virus infection. Hum Vaccin Immunother 14: 1820-31
55. Fu T M, Guan L, Friedman A, Schofield T L, Ulmer J B, Liu M A, Donnelly J J. 1999. Dose dependence of CTL precursor frequency induced by a DNA vaccine and correlation with protective immunity against influenza virus challenge. J Immunol 162: 4163-70
56. Donnelly J J, Friedman A, Martinez D, Montgomery D L, Shiver J W, Motzel S L, Ulmer J B, Liu M A. 1995. Preclinical efficacy of a prototype DNA vaccine: enhanced protection against antigenic drift in influenza virus. Nat Med 1: 583-7
57. Yu J, Tostanoski L H, Peter L, Mercado N B, McMahan K, Mahrokhian S H, Nkolola J P, Liu J, Li Z, Chandrashekar A, Martinez D R, Loos C, Atyeo C, Fischinger S, Burke J S, Slein M D, Chen Y, Zuiani A, Lelis F J N, Travers M, Habibi S, Pessaint L, Van Ry A, Blade K, Brown R, Cook A, Finneyfrock B, Dodson A, Teow E, Velasco J, Zahn R, Wegmann F, Bondzie E A, Dagotto G, Gebre M S, He X, Jacob-Dolan C, Kirilova M, Kordana N, Lin Z, Maxfield L F, Nampanya F, Nityanandam R, Ventura J D, Wan H, Cai Y, Chen B, Schmidt A G, Wesemann D R, Baric R S, Alter G, Andersen H, Lewis M G, Barouch D H. 2020. DNA vaccine protection against SARS-CoV-2 in rhesus macaques. Science 369: 806-11
58. Suschak J J, Wang S, Fitzgerald K A, Lu S. 2016. A cGAS-Independent STING/IRF7 Pathway Mediates the Immunogenicity of DNA Vaccines. J Immunol 196: 310-6
59. Suschak J J, Wang S, Fitzgerald K A, Lu S. 2015. Identification of Aim2 as a sensor for DNA vaccines. J Immunol 194: 630-6
60. Wu H, Chen Y, Liu H, Xu L L, Teuscher P, Wang S, Lu S, Dent A L. 2016. Follicular regulatory T cells repress cytokine production by follicular helper T cells and optimize IgG responses in mice. Eur J Immunol 46: 1152-61
61. Hollister K, Chen Y, Wang S, Wu H, Mondal A, Clegg N, Lu S, Dent A. 2014. The role of follicular helper T cells and the germinal center in HIV-1 gp120 DNA prime and gp120 protein boost vaccination. Hum Vaccin Immunother 10: 1985-92
62. Ulmer J B, Wahren B, Liu M A. 2006. Gene-based vaccines: recent technical and clinical advances. Trends Mol Med 12: 216-22
63. Lu S. 2011. Two is better than one. Lancet Infect Dis 11: 889-91

1. A DNA vaccine for use in a subject against SARS-CoV-2 virus infection comprising a polynucleotide sequence encoding a polypeptide of the SARS-CoV-2 virus, wherein the polynucleotide sequence is codon optimized for expression in the subject.

2. (canceled)

3. The DNA vaccine of claim 1, wherein the polypeptide comprises the receptor-binding domain (RBD) of the spike protein.

4. The DNA vaccine of claim 1, wherein the subject is a human being.

5. (canceled)

6. The DNA vaccine of claim 1, wherein the polynucleotide sequence comprises a sequence of SEQ ID NO: 3 or 4.

7. A method for preventing or treating SARS-CoV-2 virus infection in a subject comprising administering to the subject an effective amount of the DNA vaccine of claim 1.

8-12. (canceled)

13. A vaccine combination for use in a subject against SARS-CoV-2 virus infection comprising:
1) a DNA vaccine comprising a polynucleotide sequence encoding a polypeptide of the SARS-CoV-2 virus; and
2) an antigen peptide vaccine, wherein the antigen peptide is an antigen peptide of the SARS-CoV-2 virus.

14. The vaccine combination of claim 13, wherein the polynucleotide sequence is codon optimized for expression in the subject.

15. (canceled)

16. The vaccine combination of claim 13, wherein the polypeptide comprises RBD of the spike protein.

17. The vaccine combination of claim 13, wherein the subject is a human being.

18. The vaccine combination of claim 13, wherein the DNA vaccine is a plasmid constructed from plasmid pSW3891.

19. The vaccine combination of claim 13, wherein the polynucleotide sequence comprises a sequence of SEQ ID NO: 3 or 4.

20. (canceled)

21. The vaccine combination of claim 13, wherein the antigen peptide comprises RBD of the spike protein.

22. The vaccine combination of claim 13, wherein the antigen peptide is the 51 subunit of the spike protein.

23. The vaccine combination of claim 13, wherein the antigen peptide comprises an amino acid sequence of SEQ ID NO: 7 or a functional variant with sequence identity of 80% or more to SEQ ID NO: 7.

24. The vaccine combination of claim 13, wherein the DNA vaccine and the antigen peptide vaccine are co-formulated in a vaccine formulation or each formulated as a separate vaccine formulation, with a pharmaceutically acceptable vehicle.

25. The vaccine combination of claim 13, wherein the DNA vaccine and the antigen peptide vaccine are formulated as a vaccine formulation suitable for co-delivery through intramuscular injection.

26. A method for preventing or treating SARS-CoV-2 virus infection in a subject comprising administering to the subject an effective amount of the vaccine combination of claim 13.

27-37. (canceled)

38. The method of claim 26, wherein the DNA vaccine and the antigen peptide vaccine are co-administrated to the subject.

39. The method of claim 26, wherein the DNA vaccine and the antigen peptide vaccine are co-administrated to the subject at least 3 times.

40. The method of claim 26, wherein the DNA vaccine and the antigen peptide vaccine are administrated through intramuscular injection.

41-43. (canceled)
